(19) United States
(12) Patent Application Publication
(10) Pub. No.: US 2017/0199978 A1
(43) Pub. Date: Jul. 13, 2017

(54) USE OF AN EVIDENCE-BASED, TRANSLATIONAL ALGORITHM TO, INTER ALIA, ASSESS BIOMARKERS

(71) Applicant: GENOVA DIAGNOSTICS, INC., Asheville, NC (US)

(72) Inventor: Darryl Landis, Asheville, NC (US)

(21) Appl. No.: 15/327,202

(22) PCT Filed: Jul. 17, 2015
(86) PCT No.: PCT/US2015/041038

§ 371 Date: Jan. 18, 2017

Related U.S. Application Data
(60) Provisional application No. 62/026,241, filed on Jul. 18, 2014.

(51) Int. Cl. G06F 19/00; A61K 31/525; G06F 19/18

(52) U.S. Cl. CPC G06F 19/3431; G06F 19/18; A61K 31/525

(57) ABSTRACT

Described herein are methods of assessing a wide variety of physiological needs a subject (e.g., a human patient) may have as a result of an internally-driven or externally-imposed event. Aspects of the methods are computer-aided and can be used to assess a subject's need for nutritional or medicinal support. Accordingly, the invention features computer systems configured to carry out the methods described herein and computer-readable media containing program code for performing the methods. The invention also encompasses the generation of biological translation curves, and the information obtained by the present methods can be extended to include therapeutic methods that rely on complex analyses of a plurality of analytes related to a given biomarker. The invention also features pharmaceutical or physiologically acceptable compositions that are tailor-made for a given subject (e.g., a human patient) or group of subjects (e.g., a herd of livestock or crop of plants).

### CROSS-REFERENCE TO RELATED APPLICATIONS

This application claims the benefit of the priority date of U.S. Provisional Application No. 62/026,241, filed Jul. 18, 2014, the entire content of which is hereby incorporated by reference herein.

### FIELD OF THE INVENTION

The present invention relates to methods of assessing a wide variety of physiological needs a subject (e.g., a human patient) may have as a result of an internally-driven or externally-imposed event (e.g., an inadequate diet, exposure to a toxin, a recognized disease state, other medical condition, or other type of perturbation). Aspects of the methods are computer-aided and can be used to assess a subject's need for nutritional or medicinal support. Accordingly, the invention features computer systems configured to carry out the methods described herein, including methods of assessing a subject's need for nutritional or medicinal support, and computer-readable media containing program code for performing the methods. As a part of the analysis, the invention also encompasses the generation of biological translation curves, and the information obtained by the present methods can be extended to include therapeutic methods that rely on complex analyses of a plurality of analytes related to a given biomarker. In another aspect, the invention features pharmaceutical or physiologically acceptable compositions that are tailor-made for a given subject (e.g., a human patient) or group of subjects (e.g., a herd of livestock or crop of plants that are exposed to similar conditions).

### BACKGROUND

The B vitamins, which include thiamine (B1), riboflavin (B2), niacin (B3) pyridoxine (B6), folate (B9), biotin (B7) and cobalamin (B12), serve as cofactors or co-substrates for enzyme pathways throughout the body and, as such, are among the most important nutritional biomarkers. High doses of B vitamins can partially compensate for common single nucleotide polymorphisms (SNPs) that cause enzymes in these pathways to exhibit a decreased binding affinity, and hence a lower rate of reaction (Ames et al., Am. J. Clin. Nutr. 75:616-658, 2002; Ames et al., In Nutrigenomics: Discovering the Path to Personalized Nutrition 277-293, 2006). Studies of the inborn errors of metabolism (IEM) have shed light on the intimate relationship between vitamin cofactors and the enzymes that depend upon them. For instance, the administration of supra-physiologic amounts of corresponding vitamin cofactors to patients with genetic enzyme defects has been shown to at least partially restore enzymatic function (Ames, Arch. Biochem. Biophys. 423(1):227-234, 2004). Although many IEMs discussed in the literature represent extreme and/or rare examples of complete enzyme dysfunction, the same mechanism has been shown to operate in individuals with partial enzyme impairment. A good example of this is the gene encoding methylenetetrahydrofolate reductase (MTHFR), which is central to the metabolism of homocysteine and folate. A number of SNPs have been shown to affect MTHFR, and some of these are relatively common in the general population, affecting up to 20% of individuals (Leclerc et al., NCBI Bookshelf ID NBK6561). High homocysteine levels have been associated with cardiovascular disease (Wald et al., BMJ 325:1-7, 2002) and, because of this, the SNPs affecting MTHFR have perhaps been the most studied; we have seen that the significance of this mutation in relation to homocysteine levels is amplified under conditions of both folate and riboflavin depletion (Scott, Proc. Natl. Acad. Sci. USA 98(6):14754-14756, 2001; McNulty et al., Circulation 113:74-80, 2006; and Weisberg et al., Atherosclerosis 156:409-415, 2011).

### SUMMARY OF THE INVENTION

In a first aspect, the present invention features methods of generating a functional need score, which indicates whether an individual subject (or patient) is in a particular physiological state or has a particular physiological need. As described further below, these states and needs are referred to herein as “biomarkers,” and methods of assessing analytes (e.g., to generate a biological translation curve related to a corresponding biomarker) constitute a second aspect of the invention.

The methods of generating a functional need score can include the steps of: (a) providing a sample from a subject; (b) measuring, in the sample, a plurality of analytes related to a biomarker; (c) designating, for each analyte that is above or below a specified normal limit, an assigned relationship score that reflects the strength of the relationship between the analyte and the biomarker; (d) generating a total assigned relationship score by summing each of the assigned relationship scores; and (e) using the total assigned relationship score to generate a functional need score.

Once available, the total assigned relationship score can be used in various ways to generate or identify a functional need score and thereby assess the corresponding biomarker. For example, a computer system can map the total assigned relationship score, in either the form originally produced or in a further manipulated form, onto a biological translation curve in order to determine a functional need score. Alternatively, the computer system can incorporate the total assigned relationship score into an equation that solves for the functional need score. The functional need score can then be compared to a functional need scale (e.g., a numeric scale from, for example, 0 to 10 (as a matter of convenience) or a colorimetric scale) which has been demarcated to signify the limits within which a patient's functional need score translates to a normal functional need or normal physiological state (in which case intervention would not be needed) or an abnormal functional need or abnormal physiological state (in which case intervention would be considered and/or recommended). For example, a patient's functional need score may indicate a moderate or high functional need for a nutritional biomarker or a moderately or highly disturbed physiological state (e.g., pronounced dysbiosis). While we have elected to refer to moderate and high functional needs, it is to be understood that different terms (e.g., “somewhat-elevated” or “markedly-elevated”) could also certainly be used. Further, additional categories (i.e., more than the two illustrated herein as “moderate” and “high”) can also readily be defined. In one embodiment, the subject's need is categorized as exceeding a normal need by a certain percentage or fold increase.

In any embodiment, the subject can be a mammal; the sample can be a tissue, blood, serum, plasma, or urine sample; and when the sample is a urine sample, the method can optionally further include a step of measuring creatinine levels in the urine sample.

In any embodiment, the present methods can be employed to assess a biomarker that is a biological molecule. For example, the biomarker can be vitamin A (a carotinoid), vitamin E (a tocopherol), CoQ10, a plant-based antioxidant, vitamin C, α-lipoic acid, glutathione, thiamin (vitamin B1), riboflavin (vitamin B2), niacin (vitamin B3), pyridoxine (vitamin B6), biotin (vitamin B7), folic acid (vitamin B9), cobalamin (vitamin B12), manganese, molybdenum, magnesium, zinc, a nucleic acid, a lipid or fat molecule, a probiotic, a pancreatic enzyme, a marker of mitochondrial dysfunction, a molecule selectively expressed by a microbe (e.g., a bacterium, virus, or fungus), or a cancer-specific antigen. In various embodiments, the biological molecule can be assessed with regard to its level of expression, level of activity, or a post-transcriptional or post translational state (e.g., methylation).

In particular embodiments, (a) the biological molecule is CoQ10, and the plurality of analytes includes lactic acid (H), succinic acid (H), b-OH-b-methylglutaric acid (H), CoQ10 (L/H), or a combination thereof; (b) the nutritional biomarker is vitamin C, and the plurality of analytes includes cystine (H), glutathione (L), 8-OHdG (H), or a combination thereof (c) the biological molecule is riboflavin (B2), and the plurality of analytes includes pyruvic acid (H), a-ketoglutaric acid (H), succinic acid (H), adipic acid (H), suberic acid (H), kynurenic acid (H), a-ketoisovaleric acid (H), a-ketoisocaproic acid (H), a-keto-b-methylvaleric acid (H), glutaric acid (H), histidine (H), α-aminoadipic acid (H), sarcosine (H), or a combination thereof (d) the biological molecule is niacin (B3), and the plurality of analytes includes pyruvic acid (H), isocitric acid (H), a-ketoglutaric acid (L/H), malic acid (H), b-OH-b-methylglutaric acid (H), 5-OH-indolacetic acid (L/H), kynurenic acid (H), quinolinic acid (L), a-ketoisovaleric acid (H), a-ketoisocaproic acid (H), a-keto-b-methylvaleric acid (H), xanthurenic acid (L), isoleucine (L) leucine (L), lysine (L), methionine (L), phenylalanine (L), threonine (L) tryptophan (L) valine (L), alanine (L), glutamic acid (H), tyrosine (L), or a combination thereof (e) the biological molecule is cobalamin (vitamin B12), and the plurality of analytes includes lactic acid (H), succinic acid (L), 5-OH-indolacetic acid (L), formiminoglutamic acid (H), methylmalonic acid (H), histidine (H), isoleucine (H), leucine (L/H), methionine (L), phenylalanine (H), valine (H), cysteine (L/H), α-aminoadipic acid (H), cystathionine (H), ammonia (H), glycine (H), or sarcosine (H), or a combination thereof or (f) the biological molecule is magnesium, and the plurality of analytes includes lactic acid (H), citric acid (H), isocitric acid (H), 5-OH-indolacetic acid (L), phenylalanine (H), taurine (L), ammonia (H), ornithine (H), urea (L), ethanolamine (H), magnesium (L/H), or a combination thereof. The designation “(H)” indicates that the analyte is typically considered abnormal when present at higher than normally expected levels, the designation “(L)” indicates that the analyte is typically considered abnormal when present at lower than normally expected levels, and the designation (L/H) indicates that the analyte is considered abnormal when present at higher or lower levels than normally expected.

In any embodiment, the present methods can be employed to assess a biomarker that is a physiological state. In particular embodiments, the physiological state: (a) has developed following exposure to a pathogen, a microbe, a toxin, a radioactive substance, smoke, ultraviolet light, heat, or an allergen; (b) is a state of arthrosis, dysbiosis, pancreatic insufficiency, or mental illness; (c) occurs in the context of aging, a neurological disease, heart disease, vascular disease, osteoporosis, cancer, liver failure, renal failure, dysbiosis, hearing loss, vision loss, or muscle wasting; (d) occurs as an unwanted side effect of a medical treatment or medical event; or (e) is characterized by inflammation. For example, the physiological state may have developed following exposure to a toxin, and the plurality of analytes can include citric acid, cis-aconitic acid, isocitric acid, glutaric acid, a-ketophenylacetic acid, a-hydroxyisobutyric acid, orotic acid, pyroglutamic acid, lead, mercury, antimony, arsenic, cadmium, or a combination thereof. Each of the plurality of analytes is abnormal when present at abnormally high levels.

The assigned relationship score is generated from a relationship scale. In one embodiment, the relationship scale is a series of values in which the lowest value is assigned to a piece of data evidencing the weakest relationship between the analyte and the biomarker, the highest value is assigned to a piece of data evidencing the strongest relationship between the analyte and the biomarker, and the value(s) between the lowest value and the highest value is/are assigned to data evidencing a relationship between the analyte and the biomarker that is between the weakest relationship and the strongest relationship. The data may be empirically generated or may have been publicly reported.

The step of using the total assigned relationship score to generate a functional need score can include mapping the total assigned relationship score, in either the form in which it was originally produced or in a further manipulated form, onto a biological translation curve in order to determine a functional need score. In some embodiments, the total assigned relationship score is (a) divided by the potential total assigned relationship score and expressed as a fraction or percentage thereof, thereby indicating the degree of analyte abnormality; and (b) the degree of analyte abnormality is mapped onto a biological translation curve to determine the functional need score. The step of using the total assigned relationship score to generate a functional need score can include incorporating the total assigned relationship score into an equation representing a biological translation curve and solving for the functional need score. For example, the equation can be Y=[(10·X)^{Z}/(10^{Z})] or Y=[(10·X)^{Z}/(10^{Z})]·10, where Y is the functional need score, X is the total assigned relationship score divided by the total potential relationship score, and Z is a number greater than zero and less than or equal to 10. In some instances, Z is Phi (˜1.618). The functional need score can be compared to a functional need scale defining a functional need for the biomarker or an agent capable of modulating the biomarker. Where the functional need is a normal need, it may be designated as any functional need score at or below about 20% of the maximally defined functional need. A moderately elevated need may be designated as any abnormality greater than about 20% but less than 80% of the maximally defined functional need, and a high functional need may be designated as at or above 80% of the maximally defined functional need.

The assigned relationship score, total assigned relationship score, functional need score, or a biological translation curve or equation against which a degree of analyte abnormality is compared can be generated by a computer system and/or using a computer-readable medium.

Where the functional need score indicates the patient is in an undesirable physiological state or has a particular physiological need, the patient can be treated by subsequent actions as described further below. Accordingly, in a third aspect, the invention features methods of prescribing a treatment or treating a patient based on the functional need score. For example, one would administer a nutritional biomarker to a patient in the event the patient was found to have a moderately elevated or high functional need for the nutritional biomarker, or one would treat or aggressively treat a patient whose physiological state is moderately or highly perturbed. Examples of treatment regimes are provided further below. Accordingly, the invention features methods of treating a patient who is suspected of having a deficient biomarker by a method including the steps of: (a) providing a sample from the patient; (b) measuring, in the sample, a plurality of analytes related to the biomarker; (c) designating, for each analyte that is above or below a specified normal limit, an assigned relationship score that reflects the strength of the relationship between the analyte and the biomarker; (d) generating a total assigned relationship score by summing each of the assigned relationship scores; (e) using the total assigned relationship score to determine the extent to which the patient is deficient with respect to the biomarker; and (f) treating the patient according to the determined need.

The step of using the total assigned relationship score to generate a functional need score can include the steps of: (a) mapping the total assigned relationship score, in either the form in which it was originally produced or in a further manipulated form, onto a biological translation curve in order to determine a functional need score or (b) incorporating the total assigned relationship score into an equation representing the biological translation curve that solves for the functional need score. The biomarker can be a nutritional biomarker, in which case treating the patient can include altering the patient's diet or administering a dietary supplement. The biomarker can be a physiological state, in which case treating the patient can include administering a treatment that changes the physiological state toward a more desirable norm.

In another aspect, the invention features methods of developing a tool for assessing a biomarker. These methods can include generating, using a computer system, a biological translation curve exhibiting the best fit to data establishing the relationship between a degree of analyte abnormality and a functional need scale. The methods can further include establishing a relationship scale to grade the strength of the evidence for a relationship between a biomarker and a plurality of analytes. The methods can further include reviewing evidence related to the relationship between an analyte and a biomarker and generating a relationship score for each analyte within a plurality of analytes related to the biomarker.

In another aspect, the invention features a a computer system or computer-readable media containing program code configured to generate a biological translation curve.

In another aspect, the invention features methods of generating data useful in constructing a biological translation curve. The method can include the steps of: (a) providing a plurality of distinct cocktails comprising a first cocktail and an Nth cocktail, wherein, relative to one another, the first cocktail includes the lowest dosage of an active agent, the Nth cocktail includes the highest dosage of the active agent, and each cocktail between the first cocktail and the Nth cocktail comprises a dosage of the active agent titrated between the lowest dosage and the highest dosage; (b) administering each distinct cocktail to each subject in a group of subjects within a population of interest, wherein the first cocktail is administered first, the Nth cocktail is administered last, and each intervening cocktail is administered in turn at some point in time between the time the first cocktail was administered and the time the Nth cocktail was administered; and (c) obtaining biological samples from the subjects after administering each cocktail. The method can further include the step of: (d) measuring, in the biological samples, the levels of expression or activity of a plurality of analytes that are related to a biomarker that is, in turn, the active agent or affected by the active agent. The method can further include the step of: (e) assigning, to each analyte that is above or below a specified normal limit of expression or activity, an assigned relationship score that reflects the strength of the relationship between the analyte and the biomarker. The method can further include the step of: (f) using the assigned relationship scores to determine the average degree of analyte abnormality observed following administration of each of the plurality of distinct cocktails in each of the subjects. The method can further include the step of: (g) transforming the average degree of analyte abnormality into a functional need scale. The average degree of analyte abnormality observed after administering the first cocktail can be equated with a maximum functional need score, the average degree of analyte abnormality observed after administering the Nth cocktail can be equated with a minimum functional need score, and the average degrees of analyte abnormality observed after administering each cocktail between the first cocktail and the Nth cocktail can be interpolated in a linear fashion between the minimum and maximum functional need scores. Methods include any combination of these steps can further include using a computer system or computer-readable medium (e.g., to generate a biological translation curve by determining the curve best fit to a plot of the degree of biomarker-related analyte abnormality and a functional need score).

In another aspect, the invention features a computer system configured to generate a functional need score for a sample from a subject. The computer system can include: a storage medium storing: (a) measurements, from the sample, of a plurality of analytes related to a biomarker, and (b) information designating, for each analyte that is above or below a specified normal limit, an assigned relationship score that reflects the strength of the relationship between the analyte and the biomarker; and at least one processor configured to process the measurements and information designating the assigned relationship scores to generate the functional need score. The processing can include: generating a total assigned relationship score by summing each of the assigned relationship scores, and generating a functional need score using the total assigned relationship score.

In another aspect, the invention features a computer-readable medium storing software for generating a functional need score from a sample from a subject. The software can include instructions for causing a computer system to receive measurements, from the sample, of a plurality of analytes related to a biomarker and can further include instructions for causing a computer system to receive a designation, for each analyte that is above or below a specified normal limit, of an assigned relationship score that reflects the strength of the relationship between the analyte and the biomarker; generate a total assigned relationship score by summing each of the assigned relationship scores; and generate a functional need score using the total assigned relationship score.

### BRIEF DESCRIPTION OF THE DRAWINGS

FIGS. 1A, 1B, and 1C are schematic representations of data generated and organized according to the methods of the present invention. FIG. 1A is shows data related to the potential total relationship score for the biomarker B2. Eight analytes were scored for the strength of their relationship to B2 then found to be abnormally high in a sample prophetically obtained from a patient. FIG. 1B shows a representative way of organizing data used to determine the degree of abnormality in the various analytes related to B2, and FIG. 1C is a graphical representation of generating the cognate biological translation curve.

FIG. 2 illustrates a generic example of a calculation of a functional need score and a corresponding biological translation curve.

FIG. 3 illustrates the assessment of a sample for the biomarker B2.

FIG. 4 illustrates the assessment of a sample for the biomarker B3.

FIG. 5 illustrates the assessment of a sample for the biomarker B12.

FIG. 6 illustrates a personal nutritional recommendation.

FIG. 7 is a histogram illustrating a step in determining the boundaries defining normal, elevated, and high functional need for patients, where the biomarker is the biodiversity of the gut microbiome.

### DETAILED DESCRIPTION

Currently, most physicians assess a patient's nutritional needs by determining whether or not the patient has an overt clinical deficiency syndrome. Some physicians directly measure selected vitamins, particularly those in the B vitamin group, but these tests may have limited application because of the manner in which these cofactors are tightly regulated in serum or plasma. In reality, vitamin deficiency syndromes like Pellagra (a vitamin B3 deficiency) remain principally a clinical diagnosis, and “abnormal” vitamin levels have been defined crudely in such cases against overt deficiency. Several developments in the new millennium suggest that it is time to re-evaluate this area of healthcare. The sequencing of the human genome has reinforced the concept of biochemical individuality and the potential for personalized medicine, and more holistic approaches such as those taken in systems biology have reinforced our notion that existing approaches to nutrition and nutritional status testing (among other analyses) can and should be improved.

The methods described herein include new approaches for determining the status of biomarkers in general, including nutritional biomarkers. The methods ultimately rely on our understanding of the physiological process (e.g., the biochemical pathway or pathways) in which a given biomarker is active or by which it can be assessed, and an advantage of the present methods is that the analysis of any given biomarker can improve as the understanding of the physiology (e.g., the biochemical pathway or pathways) affecting the marker improves. Many biomarkers, including nutritional biomarkers, can be directly assessed. For example, one can assess their amounts or levels of activity in a sample obtained from a patient (and this can be done as a part of or as an adjunct to the present methods). However, direct assessment alone can fail to detect functional deficiencies. For example, serum measurements of vitamin B12 are notoriously inconsistent and can be inaccurately reported as low or high in certain conditions, as well as misleadingly “normal” in other cases where there is either a borderline or functional deficiency (Gaby, Nutritional Medicine 2011:91-92, 2011; see also Linder, Nutritional Biochemistry and Metabolism, with Clinical Applications 1985:300, 1985). Similarly, even though the newer practice of measuring erythrocyte folic acid is more accurate than measuring this biomarker in serum, erythrocyte folic acid is affected by vitamin B12 and iron status in the erythrocyte and is also not always a reliable metric (Gaby, Nutritional Medicine 2011:74, 2011). Instead of relying on direct measurements, the present methods include measuring, in a sample obtained from a subject, a plurality of analytes (e.g., 2-25 analytes or more (e.g., about 25-50, 50-75, 75-100, or 100-250 analytes)) related to the biomarker. The analytes can include amino acids and organic acids on either side of a biochemical reaction that is catalyzed by an enzyme that depends upon the nutritional biomarker in question. Precursors reach abnormally high concentrations when an enzymatic reaction is impaired, and the concentrations of downstream products tend to decrease. Amino acids and organic acids have been measured in blood (or components of the blood, such as plasma or serum), urine, cerebrospinal fluid (CSF), and other bodily fluids, and analytes can be measured in such samples, as well as in stool samples and tissue samples (e.g., a sample obtained from a procedure such as a biopsy or epithelial cells obtained from a cheek scraping) in the context of the present methods. Some of the relationships between an analyte and a nutritional biomarker are stronger than others, and several biomarkers can relate to a given analyte. Interpreting data from such complex and interrelated pathways in the context of an individual patient is challenging, and the present methods facilitate this.

While the present methods have been developed with the intention of assessing and treating human subjects, the subject can be any biological entity in which analytes reflect the status of a biomarker. For example, the subject can be any mammal, including a human, domesticated animal (e.g., a house cat or dog), research animal (e.g., a rodent or non-human primate), or livestock. The subject can also be a crop or member of a crop (e.g, a cultivated plant, fungus, or alga that is harvested for material consumption (e.g., for food, clothing or other textile use, livestock fodder, biofuel, or medicine). Where the subject is a plant, it can be a sugarcane, pumpkin, maize, wheat, rice, cassava, soybean, hay, potato, cotton, or tobacco plant, and the sample can be extracted from the plant as a whole or a part thereof (e.g., the sample can be an extract from the roots, rhizome, stem, leaves, flowers, fruit, or seeds).

Given the complexity of physiological processes and biochemical pathways, and given that the present methods can be widely applied to many types of animals and plants, it should be understood that some instances a given moiety may be either a biomarker or an analyte. For example, vitamin B2 can be a biomarker and, at the same time, where vitamin B2 serves to indicate the status of a different biomarker, vitamin B2 can be used, in that instance, as one of the plurality of analytes.

The term “about” is used herein to indicate a value that includes an inherent variation of error for the device or the method being employed to determine the value or plus-or-minus 10% of the value, whichever is greater.

By “analyte” we mean any substance (e.g., a biological molecule), any more complex entity (e.g. cells in tissues or present within, for example, the blood, CSF, saliva, stool, or urine (e.g., any type of leukocyte or erythrocyte)) or state (e.g., blood pressure or blood glucose level) useful in indicating the functional status of a biomarker. For example, an analyte can be a compound or molecule that serves as a substrate for a chemical reaction or that is produced by a chemical reaction in a biological pathway or system that bears some functional relationship to the biomarker in question. For example, an analyte can serve as a substrate for a chemical reaction in a metabolic pathway in which a nutritional biomarker serves as a cofactor for an enzyme. A given analyte may be related to more than one nutritional biomarker. For example, one may examine glycine as an analyte when evaluating the nutritional biomarker B12, another B vitamin (e.g., B6), or another vitamin, such as vitamin A or vitamin E.

By “assigned relationship score” we mean a relationship score of some magnitude (i.e., not zero) that is assigned to an analyte when that analyte is found to differ from a specified norm (e.g., a level of expression or activity) in a sample obtained from a patient. Where an analyte is abnormal (i.e., present at higher or lower levels than the specified norm or more or less functionally active than the specified norm), the assigned relationship score has the same value as the relationship score. Where an analyte is normal or within normal limits (i.e., present in an amount or having a level of activity within a specified norm), no relationship score is assigned to that analyte. Alternatively, and in effect, the computer system could apply no value (i.e., the assigned relationship score would be zero or otherwise negated or neutralized).

A “biological translation curve” is a linear or curvilinear plot against which a functional need score can be mapped or compared in order to determine whether a subject whose sample was tested for a plurality of analytes related to a biomarker (e.g., a nutritional biomarker) has a normal need (i.e., a need that is currently being met and does not require an intervention to improve the subject's health, prognosis for continued good health, or feeling of well being) or a need that is elevated to some degree (e.g., a moderately elevated need or a high functional need), thereby indicating a need for therapeutic manipulation of the nutritional biomarker. One of ordinary skill in the art will appreciate that linear or curvilinear plots can be represented by mathematical equations. Accordingly, the biological translation curve can also be described as a mathematical expression that describes the relationship between a plurality of analytes and a biomarker (e.g., a biomarker that has been perturbed in response to an intervention (e.g., the administration of the biomarker to a subject population as described further herein)). Thus, a functional need score can be determined either by mapping from the plot (e.g., using the axes on a graphical representation of the plot) or by solving for the functional need score using an equation describing the plot and points thereon.

By “biomarker” we mean a biological molecule or a physiological state that can be objectively evaluated by a method described herein and that serves, as a result, as an indicator of the status of an individual subject's health. Where the biomarker is a biological molecule, it can be an element, chemical compound, or more complex moiety found in vivo either through endogenous production or acquisition (e.g., consumption or exposure). The biological molecule can be organic or inorganic. For example, the biomarker can be a vitamin, a lipid, a mineral (in either elemental or compound form; e.g., calcium, iron, potassium, or zinc), a nucleic acid (e.g., a gene), a peptide (e.g., a neuropeptide), or protein (e.g., an enzyme, hormone, transcription factor, or growth factor). Where a biomarker can fluctuate depending on the subject's diet (e.g., where the biomarker is present in food or varies in the subject depending on the subject's diet), it can be further described as a nutritional biomarker. Thus, a “nutritional biomarker” is one that can, for example, be consumed (e.g., a vitamin or mineral) or is otherwise related to diet or nutritional status. Where the biomarker is a physiological state, it can be a physiological condition in a subject that arises through a mechanism internal to the subject (e.g., a recognized disease process such as cancer or other perturbation such as a failure to absorb micronutrients) or an external mechanism (e.g., exposure to a toxin, radiation, allergen, or other injurious force). Biological molecules can be evaluated in either a normal state (i.e., a state considered by prevailing medical wisdom to exist when the compound or molecule is functioning normally) or a modified state that is associated with a disease process or other perturbation (e.g., aggregated, chelated, dimerized, glycosylated, methylated, amidated, phosphorylated, or the like). In some instances, the biomarker and any subsequent treatment can be one and the same. For example, a biomarker can be a given vitamin or mineral that the subject would be recommended to consume should the present methods establish that the subject is deficient for that vitamin or mineral. In other instances, the biomarker and any subsequent treatment can be different. For example, where the biomarker is a compound or molecule that signals an adverse reaction (e.g., an adverse reaction to an allergen or toxin) the treatment could be a medication useful in treating the adverse reaction (e.g., an antihistamine, antidote, or counter-toxin).

The “functional need score” is an indicator of a physiological state or physiological need that is developed for an individual subject. The score may be a number but is not necessarily numerical; the score may be represented by, or converted to, a color, symbol or other notation.

Although the term “patient” may be used to refer to an individual who has some recognized medical condition (e.g., cancer or a mental illness), we may also use the term more generally to refer to any subject or individual regardless of their state of health. We tend to refer to a “subject” as an individual plant or animal subjected to the present methods and to a “patient” as a subject who is subsequently treated due to a detected abnormality. The methods of the invention can include a step of selecting a subject who has a suspected or recognized nutritional or medical need. Alternatively, a subject can be selected who/that is apparently healthy (e.g., a flourishing plant or animal in good health) for the purpose of monitoring the state of health and receiving early indications of perturbations (as evidenced by slightly elevated functional need). Thus, in some embodiments, a subject's medical condition is taken into account and in other embodiments the methods are practiced on either apparently healthy subjects or without regard for or other knowledge about a subject's state of health. Accordingly, the methods described herein can include or exclude a step of performing a (or another) diagnostic test or therapeutic procedure. For example, the methods of determining a functional need score can include a step of subjecting the subject to another diagnostic test (e.g., a test for a chronic condition such as diabetes, a test for endocrine function, a test to detect cancer (e.g., an imaging procedure), inflammation, or atherosclerosis, or a test to assess mental acuity or mental illness).

We may use the terms “peptide” and “protein” interchangeably to refer to polymers of amino acid residues, regardless of their length, sequence, or post-translational modification. We tend to favor the term peptide when referring to shorter polymers, such as neuropeptides or any fragments of larger, naturally-occurring or synthetic proteins, and we tend to favor the term protein when referring to full-length, naturally-occurring or synthetic versions of proteins such as enzymes, hormones, transcription factors, and growth factors.

By “relationship scale” we mean a range of values, which can be conveniently expressed as numerical values (e.g., positive integers or levels), indicating the strength of the relationship between an analyte and a biomarker. As with other indicators or scores, the relationship scale may be represented by a range of numbers, but it is not necessarily numerical. The relationship scale may be represented by, or converted to, a range of colors, symbols or other notations.

By “relationship score” we mean a value (e.g., a numerical value) that is generated based on a relationship scale for each analyte in a plurality of analytes related to a given biomarker (e.g., a nutritional biomarker).

By “total assigned relationship score” we mean a value (e.g., a number in the event the methods are carried out using numerical values) that is generated by summing the assigned relationship scores of each of a plurality of analytes related to a given biomarker (e.g., a nutritional biomarker). As noted above, there is, in effect, no assigned relationship score in some instances, as the is zero for each analyte that is present in an amount or has a level of activity within a specified norm. Thus, the assigned relationship scores for any such analytes within the plurality, in practice, do not contribute to the total assigned relationship score. For example, where analytes A, B, and C are related to nutritional biomarker X, the total assigned relationship score for nutritional biomarker X is the sum of the assigned relationship score of A, the assigned relationship score of B, and the assigned relationship score of C (remembering that if any one or more of these analytes are within normal limits, their assigned relationship score is zero). The assigned relationship score represents the strength of the relationship between an analyte and a biomarker as the relationship is understood and determined at a point in time. Accordingly, the relationship scale, the assigned relationship score, and the total assigned relationship score can change over time as and when new scientific evidence becomes available. Any given total assigned relationship score can change as a subject's condition changes over time (as the assigned relationship score depends on whether or not a given analyte is present or active within a normal range in a sample obtained from a subject).

By “total potential relationship score” we mean the sum of the relationship scores of all of the analytes in the plurality of analytes assessed, regardless of whether or not the analytes are within a designated normal range in a sample of biological material from a patient or subject.

A number of the elements described herein, including the assigned relationship score, the total assigned relationship score, the functional need score, and, in particular, the biological translation curve, can be generated by running a software program on a computer system; the values described herein can be generated by at least one processor or computer system receiving input stored on a storage medium coupled to the processor, and/or using a computer-readable medium storing software comprising instructions for performing the method when the software is executed on a computer system.

Analytes and biomarkers amenable to assessment with the present methods include chemical compounds, such as a vitamin, and lipids or lipoproteins. The lipid can be a phospholipid (e.g., as found in cell membranes), cholesterol (e.g., a low-density or high-density lipoprotein), triglyceride, sterol, or fatty acid. More specifically, the lipid can be sphingomyelin or may have a diacylglyceride structure (e.g., phosphatidate, cephalin, lecithin, phosphatidyl serine or phosphosphatidylcholine, or a phosphoinositide such as phosphatidylinositol, phosphatidylinositol phosphate, or phosphatidylinositol bis- or tri-phosphate).

Where the analyte or the biomarker is an element or mineral, it can be boron, calcium, chloride, chromium, cobalt (which is contained in vitamin B12), copper, fluorine, iodine, iron, lead, magnesium, manganese, mercury, nickel, phosphorous, potassium, selenium, silicon, sodium, sulfur, vanadium, or zinc. It will be understood that some biomarkers may be considered to be within two or more categories. For example, mercury may be present in some foods and also considered to be a toxin. In some instances, a given moiety (e.g., a compound) be either an analyte or biomarker.

Where the analyte or the biomarker is a nucleic acid, it can be a DNA sequence, as would be present in a gene, a regulatory region of a gene, or portions thereof. In other embodiments, where the biomarker is a nucleic acid it can be an RNA sequence, such as an mRNA or a microRNA.

Where the analyte or the biomarker is a peptide, it can be a neuropeptide such as galanin, enkephalin, neuropeptide Y, somatostatin, cholecystokinin, vasoactive intestinal peptide (VIP), substance P, or neurotensin. Where the analyte or the biomarker is a protein, it can be an enzyme or co-enzyme (e.g., a metabolic, digestive, or food enzyme) or a hormone (e.g., a peptide or steroid hormone). Examples of peptide hormones include amylin, anti-mullerian hormone, adiponectin, adrenocorticotropic hormone, angiotensinogen and angiotensin, antidiuretic hormone, atrial naturetic peptide, brain natriuretic peptide, calcitonin, cholecystokinin, corticotrophin-releasing hormone, cortistatin, enkephalin, endothelin, erythropoietin, follicle-stimulating hormone, galanin, gastric inhibitory peptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor, leptin, lipotropin, leuteinizing hormone, melanocyte stimulating hormone, motilin, orexin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone, thyrotropin-releasing hormone, and VIP. As with other agents described herein, a given agent may be categorized in more than one way. For example, VIP is both a peptide and a hormone. Steroid hormones include testosterone, dehydroepiandrosterone, androstenedione, dihydrotestosterone, aldosterone, estradiol, estrone, estriol, cortisol, progesterone, calcitriol, and calcidiol. Where the biomarker is a nutritional biomarker, it can further be classified as a macronutrient or micronutrient (appearing at levels most easily expressed as parts per million or less). Thus, a “nutritional biomarker” is one that can, for example, be consumed (e.g., a vitamin or mineral) or is otherwise related to diet or nutritional status.

Where the analyte or biomarker is a physiological state, it can be a physiological condition in a subject that arises through a mechanism internal to the subject (e.g., a recognized disease process such as cancer or other perturbation such as a failure to absorb micronutrients or a response to an environmental event such as drought) or an external mechanism (e.g., exposure to a toxin, radiation (e.g., ultraviolet light or nuclear radiation), allergen, or other injurious force). Biological molecules can be evaluated in either a normal state (i.e., a state considered by prevailing medical wisdom to exist when the compound or molecule is functioning normally) or a modified state that is associated with a disease process or other perturbation (e.g., aggregated, chelated, dimerized, glycosylated, methylated, amidated, phosphorylated, or the like). In some instances, the biomarker and any subsequent treatment can be one and the same. For example, a biomarker can be a given vitamin or mineral that the subject would be recommended to consume should the present methods establish that the subject is deficient for that vitamin or mineral. In other instances, the biomarker and any subsequent treatment can be different. For example, where the biomarker is a compound or molecule that signals an adverse reaction (e.g., an adverse reaction to an allergen or toxin) the treatment could be a medication useful in treating the adverse reaction (e.g., an antihistamine, antidote, or counter-toxin).

The Relationship Scale: As noted above, the relationship scale is a range of values that indicates the strength of the relationship between an analyte and a biomarker. The relationship scale can be conveniently expressed using numerical values (e.g., positive integers). However, other representations are possible. For example, the scale may be represented by, or converted to, a color, symbol or other notation. The lowest value (e.g., number) on the scale (often, but not necessarily, 1) can equate to the lowest grade of evidence that there is a relationship between the analyte and the nutritional biomarker; the highest number on the scale (which may be, but is not necessarily, 5) can equate to the highest grade of evidence that there is a relationship between the analyte and the nutritional biomarker; and the intervening numbers equate to intermediate grades of evidence, increasing from the lowest grade to the highest grade. Although it may not be as intuitive, the scale can be configured differently. For example, the lowest value may represent the highest grade of evidence. By “relationship,” we mean that observed changes in a given analyte (e.g., its level of expression or activity) correlate in a predictable fashion with pathophysiologic or therapeutic manipulation of the cognate/related biomarker. Any number of resources can be used to define the relationships of a given relationship scale. As described in the Example below, one resource is published scientific literature.

The Relationship Score. By “relationship score” we mean a value (e.g., a numerical value) that is generated from a relationship scale for each analyte related to a given biomarker (e.g., a nutritional biomarker). For example, where analyte A is related to biomarker X, and where the relationship scale is based on published scientific literature concerning the relationship between analyte A and biomarker X, one would generate a relationship score for analyte A by summing the values assigned to each piece of the scientific literature reporting a relationship between analyte A and biomarker X. Each value contributing to the relationship score will depend on the grade of the evidence reported and generally, as noted, the weaker evidence of a relationship is often assigned a lower score and progressively stronger evidence is assigned progressively higher scores. However, the relationship scale, upon which the relationship score is based, can be configured in other ways.

The Total Potential Relationship Score, the Assigned Relationship Score, and the Total Assigned Relationship Score. As noted above, the total potential relationship score is the sum of the relationship scores of all of the analytes in a given plurality of analytes. This “total” score does not depend on whether or not any of the analytes are present in an amount or active to a degree that is considered to be abnormal. For example, if analytes A, B, and C have a relationship with biomarker X, and if the relationship scores for analytes A, B, and C are 14, 5, and 4.5, respectively, then the total potential relationship score is 23.5 regardless of the actual amounts or actual activities of any of the analytes in a sample from a subject (e.g., a human patient). To the contrary, the relationship score only contributes to the assigned relationship score when the analyte is present in an amount (whether higher or lower) or active to a degree (whether more or less) that is considered to be abnormal. As a result, the assigned relationship score is highly likely to differ from one patient to another (inter-patient variability) and within a single patient over time (intra-patient variability). For example, if analytes A, B, and C have a relationship with biomarker X; if the relationship scores for analytes A, B, and C are 14, 5, and 4.5, respectively; and if only the test result for metabolite B is abnormal, the assigned relationship score for A is zero, the assigned relationship score for B is 5, and the assigned relationship score for C is zero. The total assigned relationship score is the sum of all of the assigned relationship scores in a given analysis. In the present example, the total assigned relationship score would be 5 (as only analyte B was abnormal and had an assigned relationship score of 5).

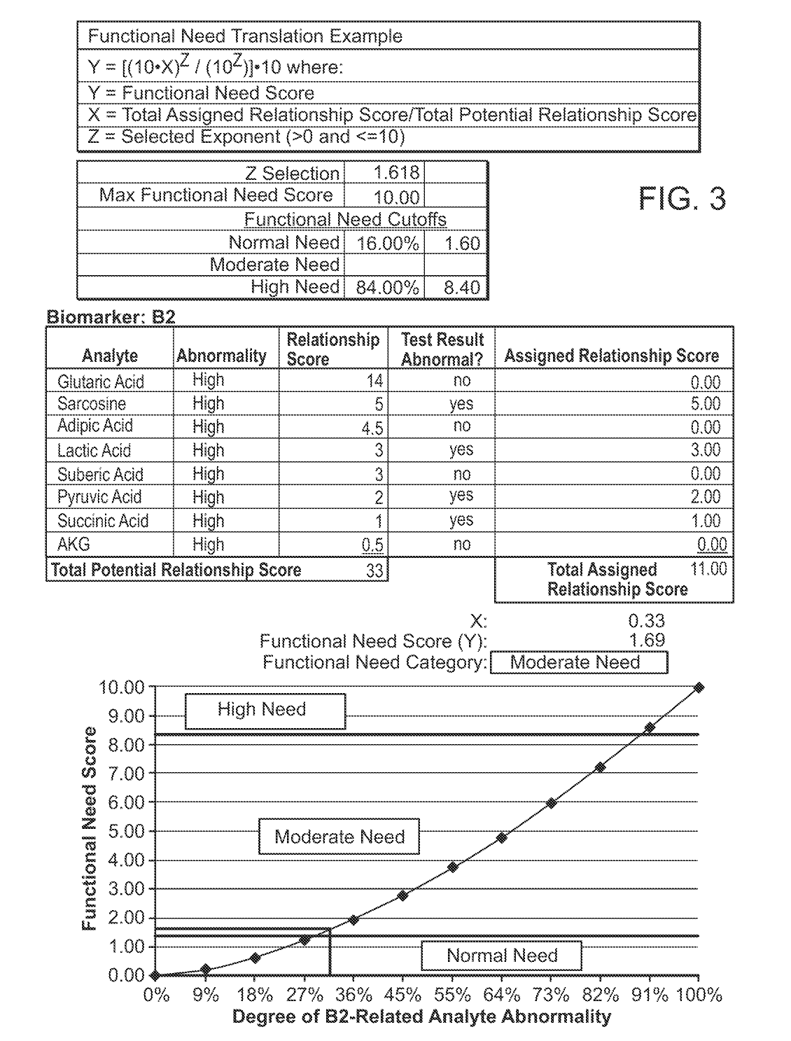

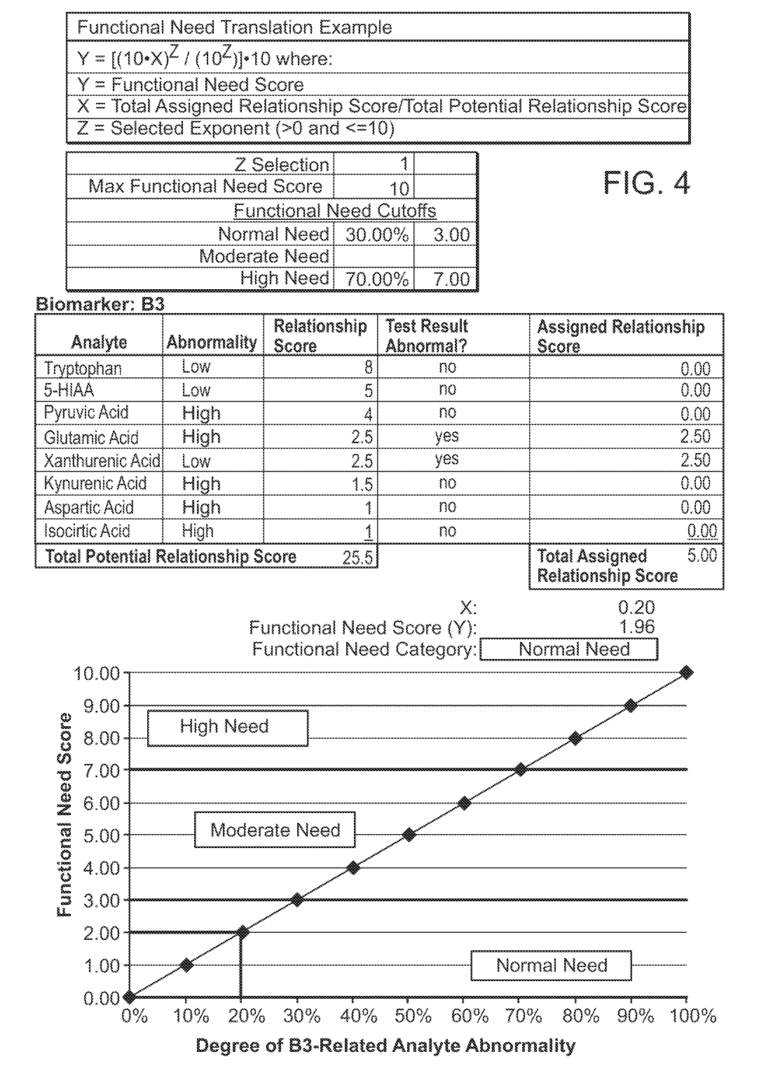

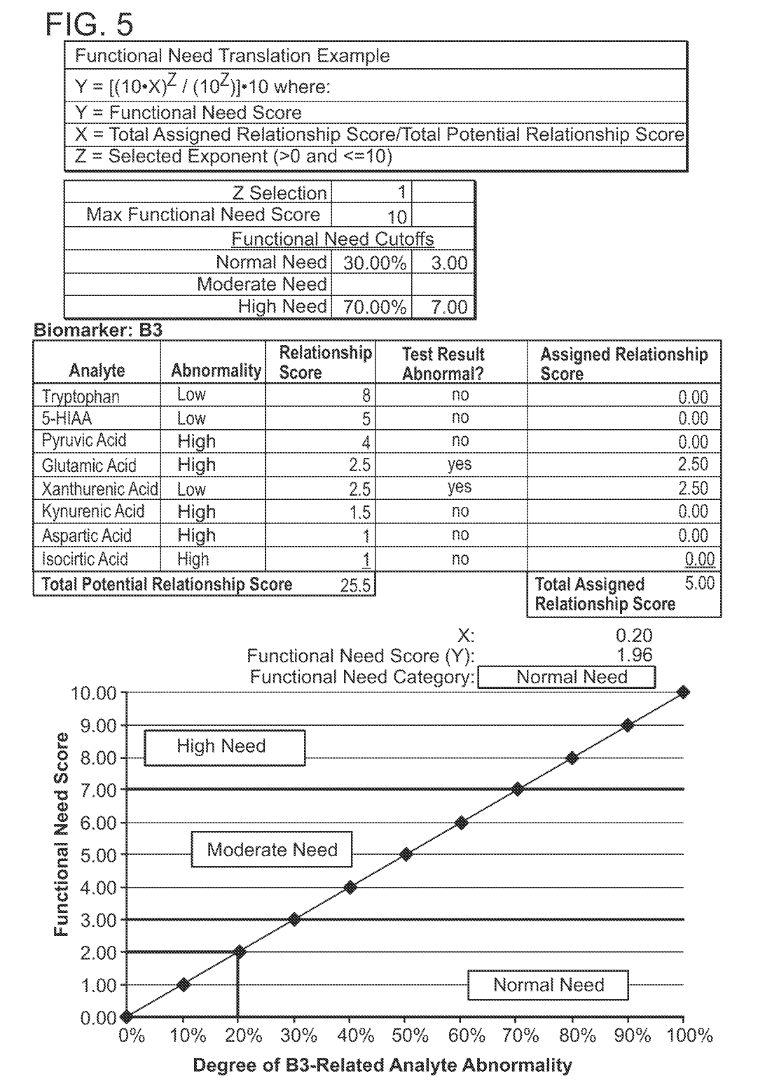

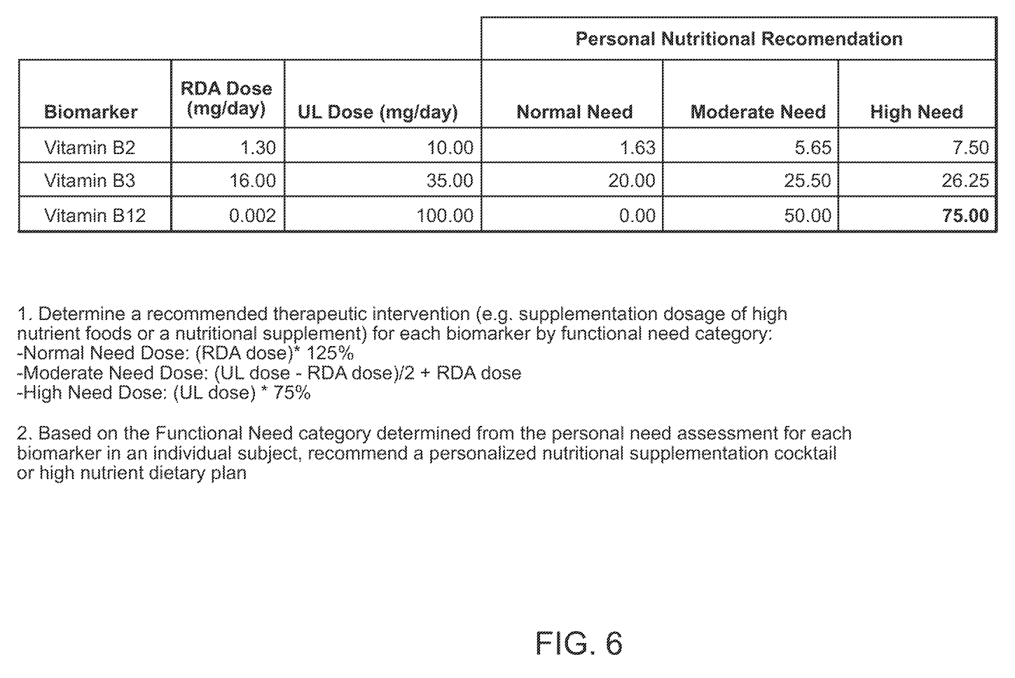

Functional Need Translation. Once available, the total assigned relationship score can be used in various ways to generate or identify a functional need score and thereby assess the corresponding biomarker. For example, the computer system can map the total assigned relationship score, in either the form produced or in a further manipulated form, onto a biological translation curve in order to determine a functional need score. For example, where the axes of the biological translation curve plot the potential total relationship score against the functional need score, one would use the total assigned relationship score in the form produced to determine, based on the curve for the biomarker in question, a subject's functional need score. As seen in FIG. 3, for example, using the analytes and relationship scores provided, the total potential relationship score for the biomarker vitamin B2 is 33. In plotting the biological translation curve, one axis could then run from zero to 33, and the total assigned relationship score of 11 could simply be used “as is” to find, based on the curve, the functional need score on the second axis. As noted, however, the total assigned relationship score can also be further manipulated, and it may be advantageous to do this for ease of comparison with standardized curves. For example, the biological translation curve in FIG. 3 plots the functional need score against the degree of B2-related analyte abnormality expressed on a scale standardized from 0-100%. As seen in FIG. 3, again using the analytes and relationship scores provided, the total potential relationship score for the biomarker vitamin B2 is 33, and the total assigned relationship score for the subject tested is 11. Thus, the total assigned relationship score is approximately 33% of the total potential relationship score, and that value of 33% is then used with the biological translation curve to find the functional need score. In both instances, of course, the score is the same (at 1.69). The total assigned relationship score can also be used in other ways to determine the functional need score. For example, it can be incorporated into an equation such as the following:

Y=[(10·X)^{Z}/(10^{Z})]·10 or Y=[(10·X)^{Z}/(10^{Z})].

where Y is the functional need score, X is the total assigned relationship score divided by the total potential relationship score, and Z is greater than zero but less than or equal to 10. In some embodiments, Z is the mathematical constant Phi, (1+√5)/2, also known as the golden ratio, which is a constant of nature. It is found repeatedly in human and animal biology, plant life and astronomy. Determining the variable Z is described further below. The equation used can vary, as it will describe the linear or curvilinear plot designated as the biological translation curve for each biomarker.

Once a functional need score is determined by such an equation, the score can be compared to a predetermined functional need scale (the scale having been developed by, for example, a method as described herein). For example, the computer system can compare the functional need score to a scale demarked in any convenient way (e.g., a numerical scale from 0-1, 1-5, 1-10, 1-100, and so forth) and demarcated to signify the extent of the subject's functional need. For example, the functional need score can be compared to a functional need scale having any number of boundaries to signify the extent of the subject's functional need. For example, boundaries can be set to designate when the functional need score signifies no need for intervention (i.e., the subject is considered to be normal or healthy) or a moderate or high need for intervention. Borderlines may similarly be designated to indicate how close a subject is to an adjacent need category. For example, functional need scores within a certain range may be designated as “high normal” to indicate that the subject's functional need is currently within a desirable, normal limit but close to (e.g., within 10% of) a value that would indicate a need for moderate intervention.

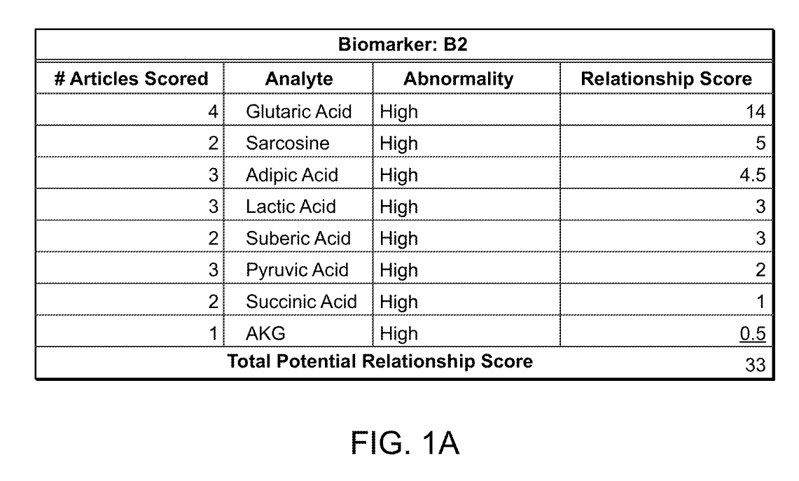

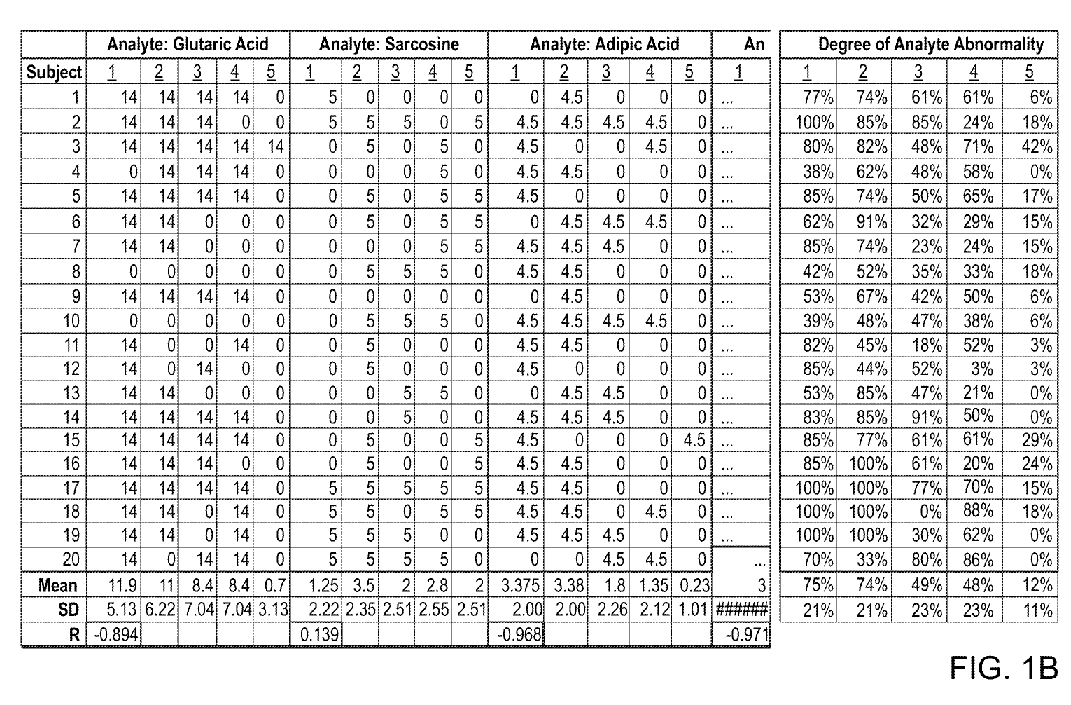

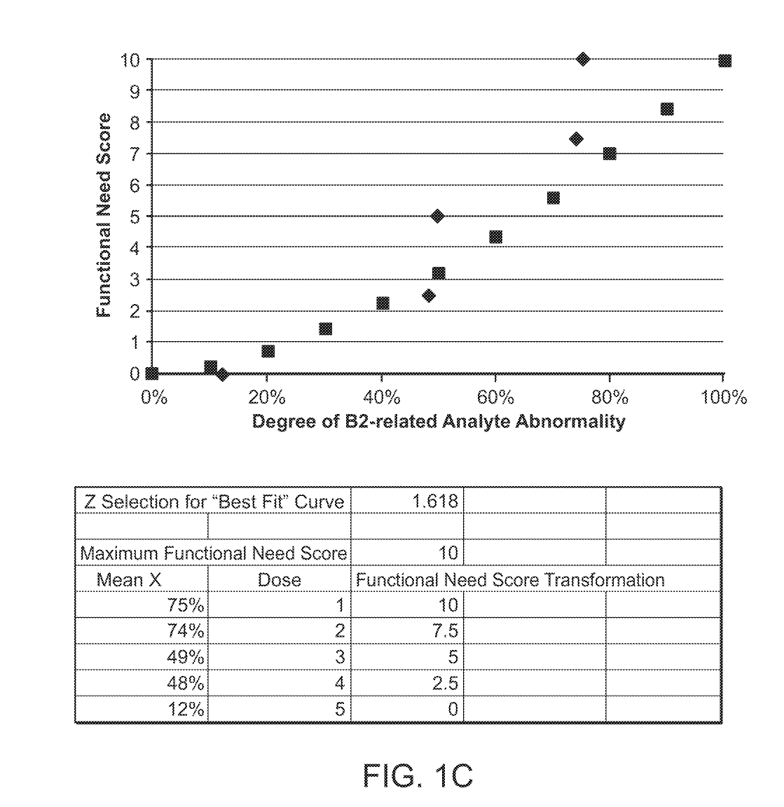

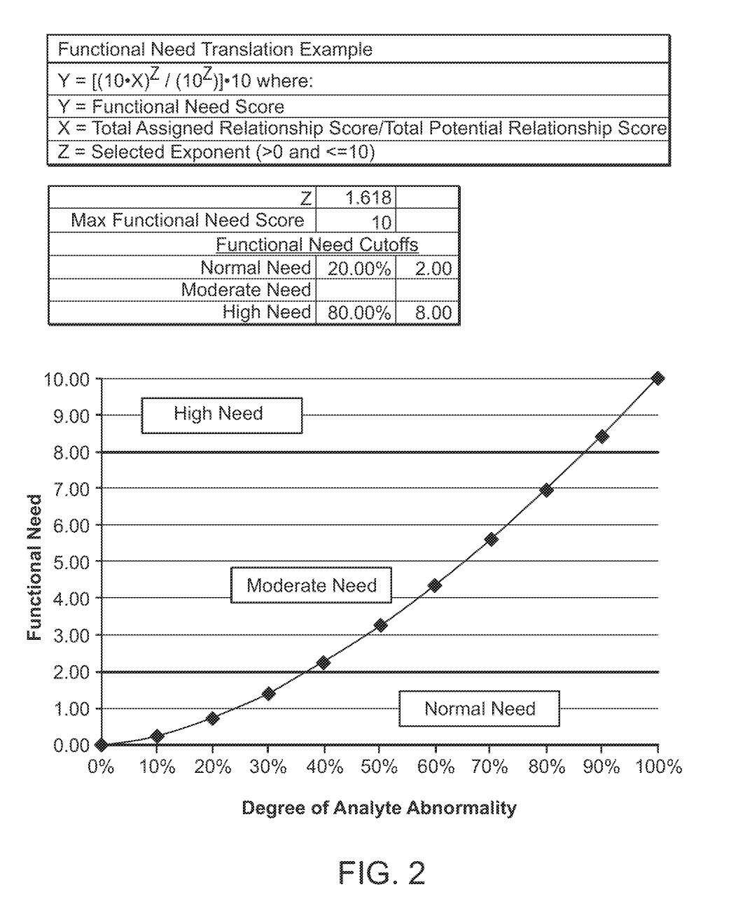

Generating Biological Translation Curves and Functional Need Scales: To generate a biological translation curve, one can begin by creating a series of supplementation cocktails that include a biomarker of interest. The number of cocktails (n) is at least three and, beyond that, the number can vary (e.g., n can be in the range of about 3-20 (e.g., 3, 4, 5, or 10) or more (e.g., 25 or 30)). The cocktails are preferably identical except for the amounts of the biomarker they include, and they can be relatively simple formulations in which a biomarker (where the biomarker is a biological molecule) is suspended in or mixed with standard, physiologically acceptable carriers (e.g., buffers and/or excipients) for oral administration (e.g., in the form of a pill, tablet, capsule, lozenge, syrup, solution, or suspension). Where a biomarker may not survive oral administration, it can be administered parenterally (e.g., topically). The cocktail designated as the “first” cocktail in the series (#1) will contain an amount of the biomarker that is the lowest amount incorporated (LL; e.g., an amount equal to the recommended daily allowance (RDA) of the biomarker). Lower amounts can be, but are not necessarily, based on the RDA; for example, the “first” cocktail can include some percentage (e.g., about 1% to about 99% of the RDA). The cocktail designated as the “last” cocktail in the series (#N) will contain an amount of the biomarker that is the highest amount incorporated in the series of cocktails (e.g., an amount equal to the tolerable upper intake level (UL)). The cocktail(s) between the first and the Nth will include an intermediate amount (or intermediate amounts) of the biomarker. The intermediate amount(s) of the biomarker can be for example, determined by the equation [(UL−LL)/(n−1)+(N−1)] (e.g., [(UL−RDA)/(n−1)+(N−1)], where N is the number of a specific cocktail and n is the total number of cocktails. One of ordinary skill in the art will recognize this equation as a means for evenly dividing a range of potential doses of a given number of cocktails and determining the exact dose for Cocktail N. Other equations and methods can be used as well to devise the series (e.g., in some instances, the dosages of the biomarkers may not be evenly divided between one cocktail and the next across the series). Using procedures known in the art, one would then assess the levels of analytes determined to be related to each biomarker for each subject in a selected reference population on a periodic (e.g., weekly) basis following sequential administration of the cocktails. The first assessment would occur after administering cocktail #1 for the first time period (e.g., after administering cocktail #1 for the first week), and the second and subsequent assessments would occur after administering the next cocktail in the series for the next period of time (e.g., the second assessment would occur after cocktail #2 was administered during the second week; the final assessment would occur after the Nth cocktail was administered after n weeks). Once the test results are known (i.e., the amounts or activities of each analyte in a sample have been determined), one would compute a total assigned relationship score for each subject at each time point and then average the results for all subjects at each time point, thereby generating a mean value (see, e.g., FIG. 1). If desired, one could discard the total assigned relationship scores that are more than, for example, three standard deviations from the raw mean, thereby generating a trimmed mean value. X (the total assigned relationship score, whether raw or trimmed, divided by the total potential relationship score; an indicator of the degree of biomarker-related analyte abnormality) can then be determined at each time point (i.e., the analytes in the plurality related to the biomarker in question can be measured following administration of each of the supplementation cocktails; the 1^{st} through the Nth). With the aid of a computer, one can then plot the cocktail number (#1 through #N) against X and, if desired, the cocktail number can be transformed into a linear functional need scaled from, for example, 1-10, where the first cocktail equates to the lowest functional need (e.g., 1) on the linear scale; the Nth cocktail equates to the highest functional need (e.g., 10) on the linear scale; and cocktails #2 through #n−1 are assigned evenly to the intervening need scores (e.g., 2-9). The computer is then configured to determine the “best fit” to a function that can be expressed in terms of any of a variety of mathematical expressions. In some embodiments, the function defining the Functional Need Score is a monotonic function of X. The mathematical expression typically contains one or more parameters that are fit to the cocktail data (e.g., the plot of cocktail number described above). For example, in some embodiments, the computer determines a value of an exponent Z in the mathematical expression Y=[(10·X)^{Z}/(10^{Z})]·10 representing a best fit to the cocktail data, where Y is the Functional Need Score (1 through 10) and X is the total assigned relationship score/total potential relationship score. Other examples of expressions can be used that include additional parameters to be determined when the computer computes the best fit, such as parameters representing a constant multiplicative factor, or a constant offset, or the expression can include higher order terms representing relatively small variations in Y as a function of X. Functional need can be categorized in any number of ways, from no perceptible need for supplementation (i.e., a normal need that is currently met) to a high need. For example, one can define functional needs on any given scale (e.g., a scale of 1-10) as normal (e.g., less than or equal to 3 on the functional need scale), moderate (e.g., 3 to 8 on the functional need scale), or high (e.g., more than 8 on the functional need scale). More generally, the boundaries defining need can be expressed as percentages along a functional need scale. For example, a functional need score within about the lower third of a functional need scale (e.g., a functional need score up to about 35% (e.g., up to about 10%, 15%, 20%, 25%, or 30% of the total functional need) may signify a normal need or state; a score within the middle third of the scale (e.g., a functional need greater than about 35% and less than about 65% (e.g., about 40%, 45%, 50%, 55%, or 60%)) may signify a moderate need or moderately perturbed state; and a score within the upper third of the scale (e.g., a functional need score within about the upper 65% or more (e.g., at least 70%, 75%, 80%, 85%, 90% or 95%) of the functional need scale may signify a high need or severely perturbed state.

In some embodiments, where the biomarker is a physiological state, the process as described herein and immediately above can be followed except that, instead of administering a series of cocktails, one could identify and group subjects that, based on objective or subjective medical assessments, are experiencing the physiological state to varying degrees of severity. For example, where the physiological state is dysbiosis, the group analogous to the least potent cocktail would have no symptoms or very mild symptoms; the group analogous to the most potent cocktail would have the most severe symptoms; and the at least one intervening group would have intermediate symptoms.

With regard to the biological translation curves, one of ordinary skill in the art will recognize curve fitting as a means to explore and determine the relationship between a set of data points. The process generally begins with a visual inspection of the scatter plot of the variables of interest. Based on this inspection, curve fitting techniques may then be used for data smoothing, data modeling, or both. By data smoothing, one is attempting to capture the important relationships between variables while omitting noise and short-term changes in the data. Data modeling attempts to identify specific mathematical relationships between the variables of interest. Once the mathematical relationships have been identified, interpolation can be used to estimate values within the observed range and extrapolation can be used to estimate values outside of the observed data range.

In the data modeling process, one evaluates the scatter plot to identify potential mathematical curves that best represent the raw data (see, e.g., Sit and Poulin-Costello, Catalog of Curves for Curve Fitting, Biometrics Information, Handbook No. 4, March 1994). Once the potential relationships have been identified, they can be programmed and then evaluated both visually and quantitatively. Often, the evaluation process will be iterative and may include comparisons of output from both linear and non-linear analytic techniques. There are many statistical packages available for data smoothing and data modeling that can be used in the context of the present methods. These include SAS, SPSS, OriginLab, SigmaPlot, Matlab, GraphPad, Microsoft Excel, etc. SAS may be preferred as it has an extensive list of procedures for performing data modeling and smoothing. These include: REG—traditional multiple linear regression; NLIN—non-linear regression; QUANTREG—quantile regression; GLM—general linear models; LOESS—median smoothing; and TPSPLINE—thin-plate spline smoothing.

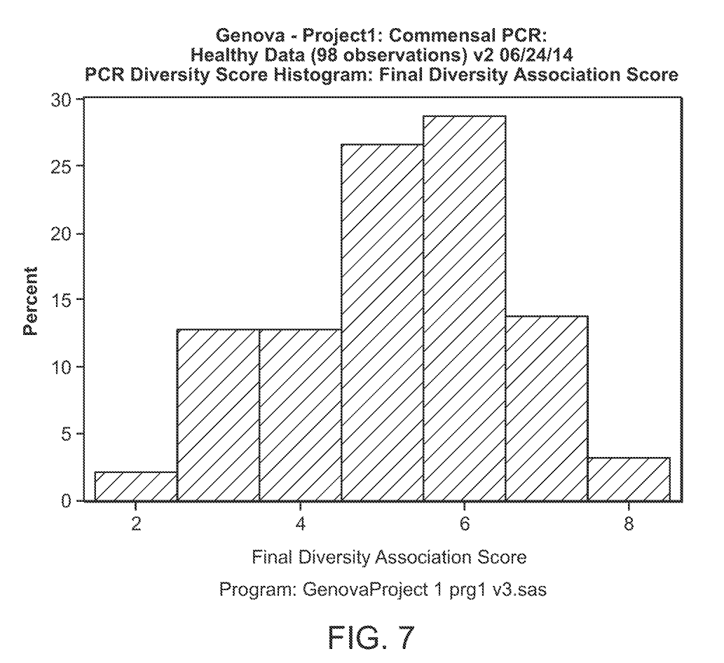

Setting the boundaries for patient need: Above, we refer to defining functional need on a given scale (e.g., a numerical scale or color scale) where, within certain limits, a patient is considered to have a certain functional need (e.g., a normal, moderate, or high functional need) for a nutritional biomarker or for a treatment to alter a physiological state. In defining the boundaries on a functional need scale, one can define criteria for a healthy reference population. For example, in determining where to place the boundaries for assessing biodiversity of the gut microbiome (a physiologic state of gut health and, as such, a biomarker), one can use the following criteria to select a healthy reference population: (1) SF12v2 scoring (e.g., a physical composite score (PCS) at average or above (50) and mental composite score (MCS) at (44) are provided by the SF12v2 wellness survey and are scored utilizing QualityMetrics software) and (2) age (e.g., a birth year between 1949-2008). Certain exclusions can also be applied to limit the healthy reference population. For example, in the preceding example, subjects can be excluded based on certain medical conditions (e.g., an active or past medical history of liver disease, autoimmune disease, gallbladder disease, inflammatory bowel disease (IBD), irritable bowel syndrome (IBS), a gastric by-pass surgery or device, diverticulosis, celiac disease, a GI-related cancer, or autism); based on active medical conditions (e.g., gallbladder disease, diabetes, stomach ulcers, non-GI cancers, and pregnancy); based on their use of certain prescription or over-the-counter medications (e.g., patients taking a medication targeting the gastrointestinal tract, an anti-viral agent, a steroid, an immune modulator, a diabetes treatment, an anti-malarial, a stimulant, or probiotics); based on the abnormal expression of biomarkers (e.g., fecal calprotection>50; EPx>7; PE-1 (pancreatic elastase)<200); and/or based on the presence of a parasitic infestation or pathogenic bacteria. After the population is defined, one can determine a functional need score for each subject in the healthy reference population, plot the distribution of values, and statistically analyze these values by, for example, calculating the standard deviation of these values. For example, in determining boundaries for assessing biodiversity of the gut microbiome (a physiologic state of gut health and, as such, a biomarker as described herein), the median Diversity Association Index (a specific implementation of a Functional Need Score for gut health), can be employed. FIG. 7 illustrates a median value of 5.4 and a standard deviation of 1.0. To set functional need boundaries, one can designate moderately abnormal degree of microbial diversity (for example) at an appropriate statistical value (e.g., 1 standard deviation value or some other appropriate statistical cutoff, such as the 95th, 50th, or 5th percentile). For example, in determining boundaries for assessing functional need with regard to the biodiversity of the gut microbiome, the 70th percentile (approximately 1 std dev higher) value of 6.0 can be selected as the boundary between a high diversity association and a moderate diversity association, and the 30^{th} percentile value of 4.4 (approximately 1 std dev lower) can be selected as the boundary between a moderate diversity association and a low diversity association.

Methods of treatment and of preparing individually tailored dosage forms: Based on the functional need score, any supplementation or treatment can then be prescribed and/or administered. For example, where a given patient has a normal dose need, a supplement including about 100-125% of the RDA of the nutritional biomarker in question could be administered; a moderate dose need could be met by, for example, (UL dose−RDA dose)/2+RDA dose; and a high dose need could be met by, for example, administering 65-85% (e.g., 75%) of the UL dose.

In any instance where the biomarker is different from the treatment (e.g., in instances where the biomarker is a physiological state), then the treatment can be dosed at different levels and the responses of related analytes can be assessed. As one of ordinary skill in the art would appreciate, this requires a relationship between the physiological state and the treatment that is well established in clinical science (since we expect that the biomarker (physiologic state) will change with administration of the appropriate treatment). For example, where the biomarker is the physiologic state of pancreatic insufficiency, the analytes can be, or can include, pancreatic elastase and fecal fat (the levels of which can be determined using available techniques). The treatment prescribed following analysis can be, for example, an orally administered pancreatic enzyme.

By analyzing a sample from a given patient (e.g., a blood, urine, and/or tissue sample) for a plurality of nutritional biomarkers, one can determine how much of each biomarker a patient needs, and supplements can be tailored to include amounts of each biomarker specifically needed by that patient. Methods of making tailored, unit dosage forms of supplements are thus within the scope of the present invention. In such methods, one would determine the amounts of a selected plurality of nutritional biomarkers required by a patient (e.g., the amounts of B vitamins) by a method as described herein and, optionally using programmed and/or automated systems, formulate a preparation (e.g., an oral, sublingual, inhalation, transmembrane, or transdermal preparation) by incorporating those amounts in a physiologically acceptable preparation (e.g., in unit dosage forms). In any of the present methods, whether generating a functional need score, generating a biological translation curve, or preparing a patient-specific formulation of nutritional biomarkers, one can analyze any combination of the following: a carotinoid (e.g., vitamin A), a tocopherol (e.g., vitamin E), CoQ10, a plant-based antioxidant, vitamin C, α-lipoic acid, glutathione, thiamin (vitamin B1), riboflavin (vitamin B2), niacin (vitamin B3), pyridoxine (vitamin B6), biotin (vitamin B7), folic acid (vitamin B9), cobalamin (vitamin B12), manganese, molybdenum, magnesium, selenium, and zinc.

The B vitamins, including B1, B2, B3, B6, B7, B9, and B12, are among the nutritional biomarkers that can be analyzed in the present methods. Vitamin B6 exists in a number of different biological forms (e.g., pyridoxine, pyridoxal, and pyridoxamine), and the metabolically active form, pyridoxal 5′-phosphate (PLP), has a role in more than 100 biochemical pathways. Pyridoxine is an important co-factor in pathways involving tryptophan and homocysteine (HCY) metabolism, among others, and analytes that can be assessed when evaluating a subject's need for pyridoxine include: xanthurenic acid, kynurenic acid, cystathionine, methionine, cysteine, ornithine, quinolinic acid, tyrosine, 5-hydroxyindoleacetic acid, serine, β-aminoisobutyric acid, β-alanine, urea, taurine, isoleucine, alanine, glutamic acid, glycine, histidine, threonine, α-aminoadipic acid, α-amino-N-butyric acid, arginine, phophoserine, cyanthonine, leucine, tryptophan, valine, or any combination thereof.

The term vitamin B12 refers to a group of cobalamins (cobalt-containing compounds). The active, coenzyme forms of vitamin B12 are adenosylcobalamin and methylcobalamin, which are created from the metabolic conversion of hydroxycobalamin. Cobalamins are important co-factors in pathways in which methylmalonic acid, isoleucine, leucine, valine, cystathionine, and homocysteine (HCY) are metabolized, and these compounds together with others such as formiminoglutamic acid, cysteine, methionine, and succinic acid can be assessed as analytes in methods of determining an individual subject's need for a nutritional biomarker that is a cobalamin.

Folate, or folic acid (vitamin B9), exists in a number of biologically active forms in the human body, including tetrahydrofolate (THF), 5-methyl-THF, and 5,10-methylene-THF. B9 is an important co-factor in pathways in which homocysteine, serotonin, methylmalonic acid, and various amino acids are metabolized, and these compounds as well as formimino-glutamic acid, cystathionine, sarcosine, 5-hydroxyindoleacetic acid, methionine, histidine, phenylalanine, serine, and glycine can be assessed as analytes in the present methods.

Thiamine (vitamin B1) deficiency is the cause of beriberi, Korsakoff's syndrome, and Wernicke's encephalopathy. B1 is important in glucose metabolism and other complex metabolic pathways (e.g., in the metabolism of dehydrogenases and amino acids, including branched chain amino acids). Most thiamine in humans is found within the erythrocyte as thiamine pyrophosphate, also known as thiamine diphosphate or TPP, and the present methods encompass analysis of B1 by measuring: pyruvic acid, α-ketoglutarate, lactic acid, α-keto-β-methylvaleric acid, α-ketoisovaleric acid, leucine, isoleucine, valine, 5-hydroxyindoleacetic acid, alanine, α-ketoadipic acid, α-aminoadipic acid, citrulline, glutamic acid, glutamine, histidine, lysine, ornithine, phenylalanine, serine, tyrosine, or a combination thereof.

Riboflavin (vitamin B2) is an important micronutrient that has a central role in human fat, ketone body, protein and carbohydrate metabolism for the generation of energy. It exists in various coenzyme states including flavin mononucleotide (FMN) and flavin adenine dinucleotide (FAD). Fewer analytes related to this nutritional biomarker may have been identified than for others described herein, but B2 is nevertheless amenable to analysis by the present methods. The related analytes include sarcosine metabolism, dehydrogenase enzymes, fatty acids, glutaric acid, adipic acid, lactic acid, suberic acid, pyruvic acid, succinic acid, and a-ketoglutarate.

In human biology, vitamin B3 is found in two forms; in the form of niacin (also known as nicotinic acid) and in the form of niacinamide (also referred to as nicotinamide). This vitamin has a key role in energy production, functioning as a precursor to NAD and to NADP, and it is also important in the metabolism of tryptophan and dehydrogenases. In clinical practice, overt clinical niacin deficiency manifests as pellagra, a serious condition that can cause dermatitis, diarrhea, dilated cardiomyopathy and dementia. Analytes related to B3 include but are not limited to tryptophan, 5-hydroxyindoleacetic acid, pyruvic acid, glutamic acid, xanthurenic acid, kynurenic acid, aspartic acid, and isocitric acid.

The final B vitamin is vitamin B7 (biotin), which functions as a coenzyme for carboxylase enzymes that have central roles in gluconeogenesis, fatty acid synthesis and in the synthesis of isoleucine and valine. Assessing this nutritional biomarker may be especially important in individual subjects known to have, or suspected of having, a nutritional deficiency syndrome (e.g., alcoholics, elderly people, those with gastric problems, epileptics, burns patients, athletes or others who subject themselves to above average exertion, and pregnant or lactating women). Analytes related to B3 include but are not limited to pyruvate, propionyl-CoA, leucine, hydroxyisovaleric acid, lactic acid, 3-hydroxypropionic acid, alanine, and glycine.

The present invention provides a framework for indicating which nutritional biomarkers (e.g., which B vitamin) are not only functionally deficient but also the severity of the deficiency. Many biochemical pathways involved are co-dependent, so a cautious approach of reassessment is always the safest one. The methods described herein can be repeated at various intervals over time with minimal inconvenience to the patient. In some instances, the present methods can be carried out on a sample obtained for another purpose (e.g., a blood sample obtained for another clinical test). Where indicated (e.g., where a patient (or individual subject) is found to have a moderate or high level of need for a given nutritional biomarker), the patient can be treated with the nutritional biomarker. One could administer, for example, the RDA of each biomarker, recognizing that a given individual may require a dose higher than that for their physiologic needs.

In view of the foregoing, and as noted above, the invention accordingly features methods of generating a functional need score, and those methods can include the steps of: (a) providing a sample from the subject; (b) measuring, in the sample, a plurality of analytes related to a biomarker; (c) designating, for each analyte that is above or below a specified normal limit, an assigned relationship score that reflects the strength of the relationship between the analyte and the biomarker; (d) generating a total assigned relationship score by summing each of the assigned relationship scores; and (e) using the total assigned relationship score to generate a functional need score. The subject can be a mammal (e.g., a human or a domesticated or farm animal) and while the sample can be one that is easily obtained (e.g., a cheek scraping or a blood, serum, plasma, or urine sample), any biological material from the patient (e.g., a tissue sample) can be used in the present methods. Similarly, it is more convenient to assess analytes in a single type of sample, but the methods are not so limited; the analytes can be assessed in different samples (e.g., one could be provided with or obtain a blood sample and a urine sample, or blood, urine, and tissue samples). Where the sample is a urine sample, the expression or activity of the biomarker can be assessed relative to the expression or activity of creatinine (see the table in Example 2). We use the term “providing” a sample to encompass any procurement of the sample. For example, providing a sample includes obtaining it from a healthcare provider who physically obtained a sample from a subject, from a patient who has obtained a sample from their own self (e.g., by scraping the mucus membrane lining the oral cavity, by pricking their skin, or by capturing a urine sample), or from a laboratory technician who obtained a sample previously collected by the healthcare provider or the patients themselves. As also noted above, the relationship score is generated from a relationship scale, which is a designated range of values (e.g., integers or fractions thereof) in which the lowest value (e.g., integer) is assigned to a piece of data evidencing the weakest relationship between the analyte and the nutritional biomarker, the highest value (e.g., integer) is assigned to a piece of data evidencing the strongest relationship between the analyte and the nutritional biomarker, and the values (e.g., integers) between the lowest value and the highest value is/are assigned to data evidencing a relationship between the analyte and the biomarker that is between the weakest relationship and the strongest relationship. The data establishing the relationships may be, but is not necessarily, publicly reported or available (e.g., the data may be gleaned from published scientific literature, patents, patent applications, textbooks, and grant proposals). In using the total assigned relationship score to generate a functional need score, the computer system can map the total assigned relationship score, in either the form in which it was originally produced or in a further manipulated form (e.g., where numerical values are converted to colors or shades of color or some other representative form), onto a biological translation curve in order to determine a functional need score. The total assigned relationship score is (a) expressed as a fraction or percentage (or an equivalent thereof) of the potential total assigned relationship score and (b) compared to a biological translation curve plotted on a graph in which a first axis represents the degree of analyte abnormality as a fraction or percentage (or an equivalent thereof) of the potential total assigned relationship score and a second axis represents the functional need score. The computer system can also use the total assigned relationship score to generate a functional need score by incorporating the total assigned relationship score into an equation that solves for the functional need score. For example, the computer system can be configured to use the equation Y=[(10·X)^{Z}/(10^{Z})]·10, where Y is the functional need score, X is the total assigned relationship score divided by the total potential relationship score, and Z is a number greater than zero and less than or equal to 10 (e.g., phi; ˜1.618). Once obtained, the functional need score is compared to a functional need scale defining a normal need, moderately elevated need, or high functional need for the biomarker or an agent directed at modulating the biomarker (e.g., where the biomarker is a physiological state). In certain embodiments, a normal need is designated as any functional need score at or below about 20% of a defined maximum functional need, a moderately elevated need is designated as any abnormality greater than about 20% but less than 80% of the maximum functional need, and a high functional need is designated as at or above 80% of the defined maximum functional need. Methods for determining the borderlines between levels of need are described further below and encompassed by the invention. In case of any doubt, any given analysis is focused throughout on a given biomarker. For example, where the biomarker is vitamin B12, the relationship scale and relationship score would relate to analytes linked to vitamin B12, and a functional need score obtained by analyzing a patient's sample for analytes related to vitamin B12 would be mapped onto a biological translation curve for vitamin B12. The outcome will determine whether any given patient has a normal, moderate, or high need for a given biomarker (or, in the event the biomarker is a physiological state, whether a given patient exhibits characteristics of a normal state, moderately perturbed state, or severely perturbed state).

In various embodiments, the biomarker can be vitamin A (a carotinoid), vitamin E (a tocopherol), CoQ10, a plant-based antioxidant (e.g., a polyphenol, such as a flavone, flavonol, or flavonone, catechin, an epicatechin, an anthrocyanidin, or a condensed tannin (proanthocyanidin)), vitamin C, α-lipoic acid, glutathione, thiamin (vitamin B1), riboflavin (vitamin B2), niacin (vitamin B3), pyridoxine (vitamin B6), biotin (vitamin B7), folic acid (vitamin B9), cobalamin (vitamin B12), manganese, molybdenum, magnesium, zinc, an essential fatty acid (e.g., alpha linoleic, eicosapentaenoic, arachidonic acids or their dietary precursor fatty acids), a probiotic or state of dysbiosis (e.g., species of Lactobacilli (esp. strains of L. brevis, L. bulgaricus, L. plantarum, L. rhamnosus, L. fermentum, L. caucasicus, L helveticus, L. lactis, L. reuteri and L. casei), Bifidobacteria (esp. strains of B. bifidum, B. longum and B. infantis), Streptococcus thermophiles and S. cremoris, S. faecium and S. infantis, and Enterococcus faecium), a digestive enzyme or, more specifically, a state of pancreatic function (e.g., a salivary, gastric, or pancreatic lipase, an amylase (e.g., pancreatic amylase), a lysozyme, haptocorrin, pepsin, trypsinogen, chymotrypsinogen, carboxypeptidase, aminopeptidase, dipeptidase, an elastase, sterol esterase, a phospholipase, a nuclease, or a sucrase, lactase, or maltase, isomaltase), a marker of mitochondrial dysfunction (e.g., lactic, pyruvic, succinic acids), a state characterized by a chemical modification (e.g., methylation, oxidation, acetylation, ubiquitination, amidation, sulfation), or toxicity (e.g., exposure to asbestos, bacterial toxins (e.g., botulinum toxin), lead, radiation, or botanical toxins (e.g., ricin and ergot alkaloids). As will be evident, some of these biomarkers are biological molecules (e.g., the vitamins, minerals, and enzymes in the foregoing list), and some of these biomarkers are physiological states (e.g., a state of toxicity). In certain embodiments, where the biomarker is a physiological state, that state may be one that: (a) has developed following exposure to a pathogen, a toxin, a radioactive substance, smoke, ultraviolet light, heat, or an allergen; (b) is a state of arthrosis, dysbiosis, pancreatic insufficiency, or mental illness (e.g., depression); (c) occurs in the context of a neurological disease, heart disease, cancer, liver failure, renal failure, or muscle wasting; (d) occurs as an unwanted side effect of a medical treatment or medical event; or (e) involves inflammation.

In the paragraphs that follow, we list analytes that can be assessed to determine a patient's functional need for certain nutritional biomarkers. We use “H” to convey that a high level of expression or activity of the analyte signifies a deficiency in the biomarker and “L” to convey that a low level of expression or activity of the analyte signifies a deficiency in the biomarker (i.e., an abnormally high or low result, respectively, would trigger the inclusion of the relationship score for the analyte in question in the total assigned relationship score). Said differently, the designation “(H)” indicates that the analyte is typically considered abnormal when present at higher than normally expected levels, the designation “(L)” indicates that the analyte is typically considered abnormal when present at lower than normally expected levels, and the designation (L/H) indicates that the analyte is considered abnormal when present at higher or lower levels than normally expected.

Where the biomarker is CoQ10, the analytes assessed can include lactic acid (H), succinic acid (H), b-OH-b-methylglutaric acid (H), CoQ10 (L/H), or a combination thereof. Where the nutritional biomarker is vitamin C, the analytes assessed can include cystine (H), glutathione (L), 8-OHdG (8-hydroxy-deoxyguanosine) (H), or a combination thereof. Where the nutritional biomarker is riboflavin (B2), the analytes assessed can include pyruvic acid (H), a-ketoglutaric acid (H), succinic acid (H), adipic acid (H), suberic acid (H), kynurenic acid (H), a-ketoisovaleric acid (H), a-ketoisocaproic acid (H), a-keto-b-methylvaleric acid (H), glutaric acid (H), histidine (H), α-aminoadipic acid (H), sarcosine (H), or a combination thereof. Where the nutritional biomarker is niacin (B3), the analytes assessed can include pyruvic acid (H), isocitric acid (H), a-ketoglutaric acid (L/H), malic acid (H), b-OH-b-methylglutaric acid (H), 5-OH-indolacetic acid (L/H), kynurenic acid (H), quinolinic acid (L), a-ketoisovaleric acid (H), a-ketoisocaproic acid (H), a-keto-b-methylvaleric acid (H), xanthurenic acid (L), isoleucine (L) leucine (L), lysine (L), methionine (L), phenylalanine (L), threonine (L) tryptophan (L) valine (L), alanine (L), glutamic acid (H), tyrosine (L), or a combination thereof. Where the nutritional biomarker is cobalamin (vitamin B12), the analytes assessed can include lactic acid (H), succinic acid (L), 5-OH-indolacetic acid (L), formiminoglutamic acid (H), methylmalonic acid (H), histidine (H), isoleucine (H), leucine (L/H), methionine (L), phenylalanine (H), valine (H), cysteine (L/H), a-aminoadipic acid (H), cystathionine (H), ammonia (H), glycine (H), sarcosine (H), or a combination thereof. Where the nutritional biomarker is magnesium, the analytes assessed can include lactic acid (H), citric acid (H), isocitric acid (H), 5-OH-indolacetic acid (L), phenylalanine (H), taurine (L), ammonia (H), ornithine (H), urea (L), ethanolamine (H), magnesium (L/H), or a combination thereof. Where the physiological state has developed following exposure to a toxin, the analytes assessed can include citric acid (H), cis-aconitic acid (H), isocitric acid (H), glutaric acid (H), a-ketophenylacetic acid (H), a-hydroxyisobutyric acid (H), orotic acid (H), pyroglutamic acid (H), lead (H), mercury (H), antimony (H), arsenic (H), cadmium (H), or a combination thereof.

As noted above, the invention features methods of treating a patient, who may or may not have been previously suspected of having a deficiency of a nutritional biomarker or an abnormal physiological state. In treating the patient according to their own functional need, one would administer (or prescribe for or counsel the patient to consume) a particular amount of a nutritional biomarker or other therapeutic agent (e.g., to treat or rebalance a disturbed physiological state). For example, where the nutritional biomarker is a B vitamin, the methods of the invention can include a step of administering (or prescribing or counseling the patient to consume): from about 0.1 mg to about 10 mg of vitamin B1 per day when the patient has a normal need for B1, from about 10 mg to about 100 mg per day when the patient has a moderate need for vitamin B1, and from about 100 mg to about 1000 mg per day when the patient has a high need for vitamin B1; from about 1 mg to about 10 mg of vitamin B2 per day when the patient has a normal need for vitamin B2, and from about 10 mg to about 100 mg per day when the patient has a moderate or high need for vitamin B2; from about about 1 mg to about 20 mg of vitamin B3 per day when the patient has a normal need for vitamin B3, from about 20 mg to about 200 mg of vitamin B3 per day when the patient has a moderate need for vitamin B3, and from about 200 mg to about 2,000 mg of vitamin B3 per day when the patient has a high need for vitamin B3; from about 0.1 mg to about 200 mg of vitamin B6 per day when the patient has a normal need for vitamin B6, about 200 mg to about 2,000 mg of vitamin B6 per day when the patient has a moderate need for vitamin B6, and from about 2 g to about 20 g of vitamin B6 per day when the patient has a high need for vitamin B6; from about 10 μg to about 200 μg of vitamin B7 per day when the patient has a normal need for vitamin B7, from about 200 μg to about 2 mg of vitamin B7 per day when the patient has a moderate need for vitamin B7, and from about 2 mg to about 20 mg of vitamin B7 per day when the patient has a high need for vitamin B7; from about 100 μg to about 1 mg vitamin B9 per day when the patient has a normal need for vitamin B9, from about 1 mg to about 10 mg vitamin B9 per day when the patient has a moderate need for vitamin B9, and from about 10 mg to about 100 mg vitamin B9 per day when the patient has a high need for vitamin B9; and from about 0.1 μg to about 100 μg of vitamin B12 per day when the patient has a normal need for vitamin B12, from about 1 mg to about 10 mg vitamin B12 per day when the patient has a moderate need for vitamin B12, and from about 10 mg to about 100 mg vitamin B12 per day when the patient has a high need for vitamin B12. The doses can be administered once a day or in divided doses (e.g., 2-4 doses) over the course of a day. An analysis of a patient's needs can be reevaluated over time (e.g., after periods of about one week, one month, or one year) by repeating a testing method as described herein, and the treatment can be adjusted accordingly. For example, if a patient who had a moderate need for vitamin B12 still has a moderate need after one month of daily supplementation with vitamin B12 (e.g., 5 mg/day) the daily intake can be doubled (e.g., to 10 mg/day).

Physiological states that can be assessed as biomarkers in the context of the present invention can be states that result from exposure to an external force, such as exposure to a pathogen (e.g., a bacterial, viral, or fungal pathogen), a microbe (e.g., a bacterium, virus, or fungus), a toxin (e.g., a heavy metal such as lead, asbestos, chromium, alcohol, an insecticide, carbon monoxide, or venom), a radioactive substance (e.g., radioisotopes such as uranium radioisotopes), smoke (including smoke inhaled from a tobacco product), ultraviolet light or heat (including light or heat of a sufficient intensity to burn the skin), or an allergen. In other instances, while the physiological state can be associated with or exacerbated by an external force, it is a state more commonly considered to arise internally. The physiological state can be a type of arthritis (e.g., osteoarthritis) dysbiosis, a mental illness (e.g., a type of anxiety or depression), a neurological disease (e.g., Alzheimer's disease or Parkinson's disease), heart disease (e.g., congestive heart failure or atherosclerosis), any type of cancer, liver failure (e.g., sclerosis) pancreatic insufficiency, renal failure, or muscle wasting. The physiological state can also be one in which there is inflammation, which may or may not be associated with any other apparent disease process.

Where the physiological state is toxic exposure, the plurality of analytes can include citric acid (H), cis-aconitic acid (H), isocitric acid (H), glutaric acid (H), a-ketophenylacetic acid (H), a-hydroxyisobutyric acid (H), orotic acid (H), pyroglutamic acid (H), lead (H), mercury (H), antimony (H), arsenic (H), cadmium (H), or a combination thereof.

Due to the nature of the methods described above, another aspect of the present invention is a method of generating a pharmaceutical or physiologically acceptable formulation for an individual patient that is specifically tailored to address a deficit discovered by the present methods of assessment. Methods of generating pharmaceutical formulations are well known in the art and can be applied in the present context.

### EXAMPLES

### Example 1

### Relationship Scales

We have developed a scale ranging from 0.5 to 5.0 points that is based on the quality of (and therefore the reliability of) published scientific literature that is relevant to the relationship between a given analyte and a given nutritional biomarker. The lowest value (here, 0.5) equates to the lowest grade of evidence, the highest value (here, 5.0) equates to the highest grade of evidence, and the interim values are accordingly assigned to evidence spanning the gap from the lowest to the highest grade. More specifically, the numerical values in this relationship scale signify the following:

| A value of: | Is assigned: |
|---|---|
| 5.0 | only once, when the relationship between an analyte and a |
| | nutritional biomarker is published in a scientific textbook as |
| | fact. |
| 4.0 | each time a relationship between an analyte and a nutritional |
| | biomarker is demonstrated in a placebo-controlled, human |
| | trial in which the analyte reverts to a normal value upon |
| | administration of the nutritional biomarker and there is a |
| | clinical response (e.g., the resolution of a sign or symptom |
| | of a disease). |
| 3.0 | each time a relationship between an analyte and a nutritional |
| | biomarker is demonstrated in a human study in which the |
| | analyte reverts to a normal value upon administration of the |
| | nutritional biomarker and the study is either placebo- |
| | controlled or there is a clinical response. |
| 2.0 | each time a relationship between an analyte and a nutritional |
| | biomarker is demonstrated in a study in which the analyte |
| | reverts to a normal value upon administration of the |
| | nutritional biomarker but the study is not placebo-controlled |
| | nor reports a clinical response (e.g., a case series report). |
| 1.0 | each time a study demonstrates a correlation between an |
| | analyte and a nutritional biomarker in urine, blood, or |
| | cerebrospinal fluid (e.g., where an analyte is measured in |
| | a sample before administration of the nutritional biomarker |
| | and its level in the sample changes after administration of |
| | the nutritional biomarker). |
| 0.5 | each time a case report (a published article describing the |
| | experience of a single patient) demonstrates a correlation |
| | between an analyte and a nutritional biomarker. |

Another example of a relationship scale that could be used in the present methods is the following:

RELATIONSHIP SCALE DEVELOPMENT--SORT* EXAMPLE

| SORT | | |
|---|---|---|
| Evidence | Relationship | |
| Level* | Scale | Criteria |
| Leve 1 | 10 | Systematic review or meta-analysis of |
| | | randomized controlled trials with consistent |
| | | findings; high-quality individual randomized |
| | | controlled trials |
| | 9 | not utilized |
| | 8 | not utilized |
| Level 2 | 7 | Systematic review or met-analysis of lower- |
| | | quality clinical trials or of studies with |
| | | inconsistent findings; lower-quality clinical |
| | | trials; cohort studies; case-control studies |
| | 6 | not utilized |
| | 5 | not utilized |
| | 4 | not utilized |
| | 3 | not utilized |
| | 2 | not utilized |
| Level 3 | 1 | consensus guidelines; extrapolations from |
| | | bench research; expert opinion; case series |
| | | or case studies |

This relationship scale is adapted from an evidence grading system described by Ebell et al. (Strength of Recommendation Taxonomy (SORT): A Patient-Centered Approach to Grading Evidence in the Medical Literature,” American Family Physician 69(3):548-556, 2004).

### Example 2

### Generating Relationship Scores

We performed a systematic review and meta-analysis of published science relating amino or organic acid levels to each of the seven B vitamins (B1, B2, B3, B6, B9, and B12). We mined electronic data bases with search terms including the names of each vitamin, the recognized clinical deficiency state (e.g. Pellagra), organic acids, amino acids and the measurement thereof. The collected studies were further hand searched for relevant references listed in the bibliography. Textbooks were included in the search. Each study was fully evaluated with respect to the strength of the scientific evidence substantiating a relationship between the organic or amino acid biomarker and the vitamin. Each study was scored on the 0.5-5.0 point Relationship Scale described in Example 1. For B2, B3, and B12, by way of example:

Nutritional Biomarker: B2

| | | | Relationship |
|---|---|---|---|
| # Articles Scored | Metabolite | Abnormality | Score |
| 4 | Glutaric Acid | High | 14 |
| 2 | Sarcosine | High | 5 |
| 3 | Adipic Acid | High | 4.5 |
| 3 | Lactic Acid | High | 3 |
| 2 | Suberic Acid | High | 3 |
| 3 | Pyruvic Acid | High | 2 |
| 2 | Succinic Acid | High | 1 |
| 1 | AKG | High | 0.5 |

| | |
|---|---|
| Total Potential Relationship Score | 33 |

Nutritional Biomarker: B3

| | | | Relationship |
|---|---|---|---|
| # Articles Scored | Metabolite | Abnormality | Score |
| 4 | Tryptophan | Low | 8 |
| 5 | 5-HIAA | Low | 5 |
| 4 | Pyruvic Acid | High | 4 |
| 3 | Glutamic Acid | High | 2.5 |
| 3 | Xanthurenic Acid | Low | 2.5 |
| 3 | Kynurenic Acid | High | 1.5 |
| 2 | Aspartic Acid | High | 1 |
| 2 | Isocritric Acid | High | 1 |

| | |
|---|---|
| Total Potential Relationship Score | 25.5 |

Nutritional Biomarker: B12

| | | | Relationship |
|---|---|---|---|
| # Articles Scored | Metabolite | Abnormality | Score |
| 10 | MMA | High | 55 |
| 6 | Cystathione | High | 13.5 |
| 5 | FIGLU | High | 8.5 |
| 3 | Cysteine | High | 5 |
| 6 | Methionine | Low | 4.5 |
| 5 | Valine | High | 3 |
| 2 | Succinic Acid | Low | 1 |
| 2 | Isoleucine | High | 1 |
| 2 | Leucine | Low | 1 |

| | |
|---|---|
| Total Potential Relationship Score | 92.5 |

As noted in the tables above, and as discussed herein, an analyte taken into consideration when determining the possible need for a nutritional biomarker may be present in a sample at either abnormally high or abnormally low levels (or may have an activity, even at normal levels of expression, that is abnormally high or abnormally low). One of ordinary skill in the art is readily able to set such levels and/or determine whether a given analyte is above or below a specified limit. For example, the following table indicates reference ranges of selected amino and organic acids, and any combination of the analytes shown in the table below can be analyzed or measured in the context of the present invention. The values provided in the table can serve to define or help define a normal limit, and the same approach can be used when other analytes are in question.

| Analyte | Level in urine | | Level in plasma | |
|---|---|---|---|---|
| Adipic acid | ≦5 | mmol/mol creatinine^{a} | 200-483 | μmol/L^{b} |
| Alanine | 17-266 | mmol/mol creatinine^{b} | | |
| α-aminoadipic acid | ≦11 | mmol/mol creatinine^{b} | ≦2 | μmol/L^{b} |
| α-amino-N-butyric | | | 1.25-3.50 | μmol/dL^{a} |
| α-ketoadipic acid | ≦2.1 | mmol/mol creatinine^{a} | | |
| α-keto-β-methylvaleric acid | | | ≦2.3 | dL^{a} |
| α-ketoglutaric acid | 12-55 | mmol/mol creatinine^{a} | | |
| α-ketoisocaproic acid | | | ≦0.91 | dL^{a} |
| α-ketoisovaleric acid | | | ≦0.85 | dL^{a} |
| Arginine | ≦5 | mmol/mol creatinine^{b} | 43-407 | μmol/L^{b} |
| Aspartic Acid | ≦2 | mmol/mol creatinine^{b} | 1-4 | μmol/L^{b} |
| β-alanine | ≦10 | mmol/mol creatinine^{b} | ≦5 | μmol/L^{b} |
| β-aminoisobutyric acid | ≦88 | mmol/mol creatinine^{b} | ≦1 | μmol/L^{b} |
| Citrulline | ≦2 | mmol/mol creatinine^{b} | 16-51 | μmol/L^{b} |
| Cystathione | ≦9 | μmol/mol creatinine^{b} | ≦1 | mmol/L^{b} |
| Cystathionine | ≦9 | μmol/mol creatinine^{b} | ≦1 | mmol/L^{b} |
| Cysteine | | | 4.6-8.0 | μmol/dL^{a} |
| FIGLU | ≦1.8 | mmol/mol creatinine^{a} | | |
| Glutamic acid | ≦3 | mmol/mol creatinine^{b} | 10-97 | μmol/L^{b} |
| Glutamine | 21-182 | mmol/mol creatinine^{b} | 428-747 | μmol/L^{b} |
| Glutaric acid | ≦0.92 | mmol/mol creatinine^{a} | | |
| Glycine | ≦330 | mmol/mol creatinine^{b} | 122-322 | μmol/L^{b} |
| 5HIAA | 1.8-8.6 | mmol/mol creatinine^{a} | | |
| 3-hydroxyisovaleric acid | ≦38 | mmol/mol creatinine^{a} | | |
| 3-hydroxypropionic acid | 6-23 | mmol/mol creatinine^{a} | | |
| Histidine | 17-266 | mmol/mol creatinine^{b} | 60-109 | μmol/L^{b} |
| Kynurenic Acid | ≦9.2 | mmol/mol creatinine^{a} | | |
| Isocitric Acid | 38-97 | mmol/mol creatinine^{a} | | |
| Isoleucine | ≦3 | mmol/mol creatinine^{b} | 34-981 | μmol/L^{b} |
| Lactic Acid | | | 4-16 | mg/dL^{b} |
| Leucine | ≦6 | mmol/mol creatinine^{b} | 73-182 | μmol/L^{b} |
| Lysine | 3-59 | mmol/mol creatinine^{b} | 119-233 | μmol/L^{b} |
| MMA | | | 87-318 | nmol/L^{b} |
| Methionine | ≦2 | mmol/mol creatinine^{b} | 16-34 | μmol/L^{b} |
| Ornithine | ≦4 | mmol/mol creatinine^{b} | 27-83 | μmol/L^{b} |
| Phenylalanine | 2-9 | mmol/mol creatinine^{b} | 40-74 | μmol/L^{b} |
| Phosphoserine | | | 0.31-0.74 | μmol/dL^{a} |
| Pyruvic Acid | 12-39 | mmol/mol creatinine^{a} | | |
| Quinolinic acid | ≦11.6 | mmol/mol creatinine^{a} | | |
| Sarcosine | ≦69 | μmol/mol creatinine^{b} | ≦4 | μmol/L^{b} |
| Serine | 10-71 | mmol/mol creatinine^{b} | 65-138 | μmol/L^{b} |
| Suberic Acid | ≦4.2 | mmol/mol creatinine^{a} | | |
| Succinic Acid | 0.8-10.4 | mmol/mol creatinine^{a} | | |
| Taurine | ≦232 | mmol/mol creatinine^{b} | 31-102 | μmol/L^{b} |
| Threonine | 4-46 | mmol/mol creatinine^{b} | 67-198 | μmol/L^{b} |
| Tryptophan | 2-14 | mmol/mol creatinine^{b} | 40-911 | μmol/L^{b} |
| Tyrosine | 38-96 | mmol/mol creatinine^{b} | 3-19 | μmol/L^{b} |
| Urea Nitrogen (BUN) | | | 7-25 | mg/dL^{b} |
| Valine | 2-5 | mmol/mol creatinine^{b} | 132-313 | μmol/L^{b} |
| Xanthurenic acid | ≦1.07 | mmol/mol creatinine^{a} | | |

^{a}Genova Diagnostics
^{b}Quest Diagnostics Laboratory

### Example 3

### Generating A Biological Translation Curve

Referring to FIG. 1A, we developed relationship scores for eight analytes related to the biomarker B2 (glutaric acid, sarcosine, adipic acid, lactic acid, suberic acid, pyruvic acid, succinic acid, and AKG (α-ketoglutarate)). The number of articles scored is indicated in the first column, and the relationship score is indicated in the last column. As current medical literature indicates that each of the eight analytes are present at abnormally high levels when vitamin B2 is deficient, we have indicated the “abnormality” for each as “high.” Summing the relationship scores for the analytes provides for a total potential relationship score of 33.

In FIG. 1B, we show how to calculate the degree of analyte abnormality, for each of the eight analytes (the graph is truncated on the right-hand side; hypothetical data for glutaric acid, sarcosine, and adipic acid are shown) at five points in time for 20 subjects. In this prophetic example, the subjects would have been treated with a set of cocktails with varying amounts of B2 (as generally described in the Detailed Description). Upon measurement of each analyte in a sample (e.g., a blood sample) obtained at the ends of the treatments, the relationship score shown in FIG. 1A would be assigned in the event the analyte is determined to be higher than an identified level. Using a computer, the assigned relationship scores are tallied and divided by the total potential relationship score in order to assess the degree of analyte abnormality at each of the five points in time, and these percentages are then averaged over all of the 20 subjects that would have been tested at each point in time. More specifically, and by way of example, if glutaric acid, sarcosine, lactic acid, pyruvic acid, succinic acid, and AKG were determined to be abnormally high in Subject 1 at the first time point (as illustrated in part in FIG. 1B), the degree of analyte abnormality in the Subject, at that time point, would be 77% ((14+5+3+2+1+0.5)/33=0.77). The degree of analyte abnormality is shown in FIG. 1B (continued), and upon averaging the degree of abnormality across the 20 hypothetical subjects, there is 75% abnormality at the first time point; 74% abnormality at the second time point; 49% abnormality at the third time point; 48% abnormality at the fourth time point; and 12% abnormality at the fifth time point. This data is plotted as shown by the diamond shapes in FIG. 1C, and a computer is used to find the best fit; see the line demarked by the square shapes in FIG. 1C. By plotting the degree of B2-related analyte abnormality against a functional need score arbitrarily scored from 1-10, this biological translation curve can now be used to determine the functional need score for vitamin B2 for any subject at any time.

What is claimed is:

1. A method of generating a functional need score, the method comprising:
(a) providing a sample from a subject;
(b) measuring, in the sample, a plurality of analytes related to a biomarker;
(c) designating, for each analyte that is above or below a specified normal limit, an assigned relationship score that reflects the strength of the relationship between the analyte and the biomarker;
(d) generating a total assigned relationship score by summing each of the assigned relationship scores; and
(e) using the total assigned relationship score to generate a functional need score.

2. The method of claim 1, wherein the subject is a mammal; the sample is a blood, serum, plasma, or urine sample; and wherein, when the sample is a urine sample the method optionally further comprises a step of measuring creatinine levels in the urine sample.

3. The method of claim 1, wherein the biomarker is a biological molecule.

4. The method of claim 3, wherein the biological molecule is vitamin A (a carotinoid), vitamin E (a tocopherol), CoQ10, a plant-based antioxidant, vitamin C, α-lipoic acid, glutathione, thiamin (vitamin B1), riboflavin (vitamin B2), niacin (vitamin B3), pyridoxine (vitamin B6), biotin (vitamin B7), folic acid (vitamin B9), cobalamin (vitamin B12), manganese, molybdenum, magnesium, zinc, a nucleic acid, a lipid or fat molecule, a probiotic, a pancreatic enzyme, a marker of mitochondrial dysfunction, a molecule selectively expressed by a microbe, or a cancer-specific antigen, wherein the biological molecule is optionally assessed with regard to level of expression, level of activity, or a post-transcriptional or post translational state.

5. The method of claim 4, wherein
(a) the biological molecule is CoQ10, and the plurality of analytes comprises lactic acid (H), succinic acid (H), b-OH-b-methylglutaric acid (H), CoQ10 (L/H), or a combination thereof;
(b) the nutritional biomarker is vitamin C, and the plurality of analytes comprises cystine (H), glutathione (L), 8-OHdG (H), or a combination thereof;
(c) the biological molecule is riboflavin (B2), and the plurality of analytes comprises pyruvic acid (H), a-ketoglutaric acid (H), succinic acid (H), adipic acid (H), suberic acid (H), kynurenic acid (H), a-ketoisovaleric acid (H), a-ketoisocaproic acid (H), a-keto-b-methylvaleric acid (H), glutaric acid (H), histidine (H), a-aminoadipic acid (H), sarcosine (H), or a combination thereof;
(d) the biological molecule is niacin (B3), and the plurality of analytes comprises pyruvic acid (H), isocitric acid (H), a-ketoglutaric acid (L/H), malic acid (H), b-OH-b-methylglutaric acid (H), 5-OH-indolacetic acid (L/H), kynurenic acid (H), quinolinic acid (L), a-ketoisovaleric acid (H), a-ketoisocaproic acid (H), a-keto-b-methylvaleric acid (H), xanthurenic acid (L), isoleucine (L) leucine (L), lysine (L), methionine (L), phenylalanine (L), threonine (L) tryptophan (L) valine (L), alanine (L), glutamic acid (H), tyrosine (L), or a combination thereof;
(e) the biological molecule is cobalamin (vitamin B12), and the plurality of analytes comprises lactic acid (H), succinic acid (L), 5-OH-indolacetic acid (L), formiminoglutamic acid (H), methylmalonic acid (H), histidine (H), isoleucine (H), leucine (L/H), methionine (L), phenylalanine (H), valine (H), cysteine (L/H), α-aminoadipic acid (H), cystathionine (H), ammonia (H), glycine (H), sarcosine (H), or a combination thereof; or
(f) the biological molecule is magnesium , and the plurality of analytes comprises lactic acid (H), citric acid (H), isocitric acid (H), 5-OH-indolacetic acid (L), phenylalanine (H), taurine (L), ammonia (H), ornithine (H), urea (L), ethanolamine (H), magnesium (L/H), or a combination thereof;
the designation “(H)” indicating that the analyte is typically considered abnormal when present at higher than normally expected levels, the designation “(L)” indicating that the analyte is typically considered abnormal when present at lower than normally expected levels, and the designation (L/H) indicating that the analyte is considered abnormal when present at higher or lower levels than normally expected.

6. The method of claim 1, wherein the biomarker is a physiological state.

7. The method of claim 6, wherein the physiological state
(a) has developed following exposure to a pathogen, a microbe, a toxin, a radioactive substance, smoke, ultraviolet light, heat, or an allergen;
(b) is a state of arthrosis, dysbiosis, pancreatic insufficiency, or mental illness;
(c) occurs in the context of aging, a neurological disease, heart disease, vascular disease, osteoporosis, cancer, liver failure, renal failure, dysbiosis, hearing loss, vision loss, or muscle wasting;
(d) occurs as an unwanted side effect of a medical treatment or medical event; or
(e) is characterized by inflammation.

8. The method of claim 7, wherein the physiological state has developed following exposure to a toxin, and the plurality of analytes comprises citric acid, cis-aconitic acid, isocitric acid, glutaric acid, a-ketophenylacetic acid, a-hydroxyisobutyric acid, orotic acid, pyroglutamic acid, lead, mercury, antimony, arsenic, cadmium, or a combination thereof, and wherein each of the plurality of analytes is abnormal when present at abnormally high levels.

9. The method of claim 1, wherein the assigned relationship score is generated from a relationship scale.

10. The method of claim 9, wherein the relationship scale is a series of values, in which the lowest value is assigned to a piece of data evidencing the weakest relationship between the analyte and the biomarker, the highest value is assigned to a piece of data evidencing the strongest relationship between the analyte and the biomarker, and the value(s) between the lowest value and the highest value is/are assigned to data evidencing a relationship between the analyte and the biomarker that is between the weakest relationship and the strongest relationship.

11. The method of claim 10, wherein the data have been publicly reported.

12. The method of claim 1, wherein the step of using the total assigned relationship score to generate a functional need score comprises mapping the total assigned relationship score, in either the form in which it was originally produced or in a further manipulated form, onto a biological translation curve in order to determine a functional need score.

13. The method of claim 12, wherein the total assigned relationship score is
(a) divided by the potential total assigned relationship score and expressed as a fraction or percentage thereof, thereby indicating the degree of analyte abnormality; and
(b) the degree of analyte abnormality is mapped onto a biological translation curve to determine the functional need score.

14. The method of claim 1, wherein the step of using the total assigned relationship score to generate a functional need score comprises incorporating the total assigned relationship score into an equation representing a biological translation curve and solving for the functional need score.

15. The method of claim 14, wherein the equation is Y=[(10·X)^{Z}/(10^{Z})] or Y=[(10·X)^{Z}/(10^{Z})]·10, where Y is the functional need score, X is the total assigned relationship score divided by the total potential relationship score, and Z is a number greater than zero and less than or equal to 10.

16. The method of claim 15, wherein Z is Phi (˜1.618).

17. The method of claim 14, wherein the functional need score is compared to a functional need scale defining a functional need for the biomarker or an agent capable of modulating the biomarker.

18. The method of claim 17, wherein the functional need is a normal need designated as any functional need score at or below about 20% of the maximally defined functional need, a moderately elevated need designated as any abnormality greater than about 20% but less than 80% of the maximally defined functional need, or a high functional need designated as at or above 80% of the maximally defined functional need.

19. The method of claim 1, wherein the assigned relationship score, total assigned relationship score, functional need score or a biological translation curve or equation against which a degree of analyte abnormality is compared is generated by a computer system and/or using a computer-readable medium.

20. A method of treating a patient who is suspected of having a deficient biomarker, the method comprising:
(a) providing a sample from the patient;
(b) measuring, in the sample, a plurality of analytes related to the biomarker;
(c) designating, for each analyte that is above or below a specified normal limit, an assigned relationship score that reflects the strength of the relationship between the analyte and the biomarker;
(d) generating a total assigned relationship score by summing each of the assigned relationship scores;
(e) using the total assigned relationship score to determine the extent to which the patient is deficient with respect to the biomarker; and
(f) treating the patient according to the determined need.

21. The method of claim 20, wherein the step of using the total assigned relationship score to generate a functional need score comprises (a) mapping the total assigned relationship score, in either the form in which it was originally produced or in a further manipulated form, onto a biological translation curve in order to determine a functional need score or (b) incorporating the total assigned relationship score into an equation representing the biological translation curve that solves for the functional need score.

22. The method of claim 20, wherein the biomarker is a nutritional biomarker and treating the patient comprises altering the patient's diet or administering a dietary supplement.

23. The method of claim 20, wherein the biomarker is a physiological state and treating the patient comprises administering a treatment that changes the physiological state toward a more desirable norm.

24. A method of developing a tool for assessing a biomarker, the method comprising generating, using a computer system, a biological translation curve exhibiting the best fit to data establishing the relationship between a degree of analyte abnormality and a functional need scale.

25. A method of claim 24, further comprising establishing a relationship scale to grade the strength of the evidence for a relationship between a biomarker and a plurality of analytes.

26. The method of claim 25, further comprising reviewing evidence related to the relationship between an analyte and a biomarker and generating a relationship score for each analyte within a plurality of analytes related to the biomarker.

27. A computer system or computer-readable media containing program code configured to generate a biological translation curve.

28. A method of generating data useful in constructing a biological translation curve, the method comprising:
(a) providing a plurality of distinct cocktails comprising a first cocktail and an Nth cocktail, wherein, relative to one another, the first cocktail includes the lowest dosage of an active agent, the Nth cocktail includes the highest dosage of the active agent, and each cocktail between the first cocktail and the Nth cocktail comprises a dosage of the active agent titrated between the lowest dosage and the highest dosage;
(b) administering each distinct cocktail to each subject in a group of subjects within a population of interest, wherein the first cocktail is administered first, the Nth cocktail is administered last, and each intervening cocktail is administered in turn at some point in time between the time the first cocktail was administered and the time the Nth cocktail was administered; and
(c) obtaining biological samples from the subjects after administering each cocktail.

29. The method of claim 28, further comprising the step of:
(d) measuring, in the biological samples, the levels of expression or activity of a plurality of analytes that are related to a biomarker that is, in turn, the active agent or affected by the active agent.

30. The method of claim 29, further comprising the step of:
(e) assigning, to each analyte that is above or below a specified normal limit of expression or activity, an assigned relationship score that reflects the strength of the relationship between the analyte and the biomarker.

31. The method of claim 30, further comprising the step of:
(f) using the assigned relationship scores to determine the average degree of analyte abnormality observed following administration of each of the plurality of distinct cocktails in each of the subjects.

32. The method of claim 31, further comprising the step of:
(g) transforming the average degree of analyte abnormality into a functional need scale.

33. The method of claim 32, wherein the average degree of analyte abnormality observed after administering the first cocktail is equated with a maximum functional need score, the average degree of analyte abnormality observed after administering the Nth cocktail is equated with a minimum functional need score, and the average degrees of analyte abnormality observed after administering each cocktail between the first cocktail and the Nth cocktail are interpolated in a linear fashion between the minimum and maximum functional need scores.

34. The method of any of claims 28-33, further comprising using a computer system or computer-readable medium to generate a biological translation curve by determining the curve best fit to a plot of the degree of biomarker-related analyte abnormality and a functional need score.

35. A computer system configured to generate a functional need score for a sample from a subject, the computer system comprising:
a storage medium storing: (a) measurements, from the sample, of a plurality of analytes related to a biomarker, and (b) information designating, for each analyte that is above or below a specified normal limit, an assigned relationship score that reflects the strength of the relationship between the analyte and the biomarker; and
at least one processor configured to process the measurements and information designating the assigned relationship scores to generate the functional need score.

36. The method of claim 35, wherein the processing includes:
generating a total assigned relationship score by summing each of the assigned relationship scores, and
generating a functional need score using the total assigned relationship score.

37. A computer-readable medium storing software for generating a functional need score from a sample from a subject, the software comprising instructions for causing a computer system to receive measurements, from the sample, of a plurality of analytes related to a biomarker.

38. The computer-readable medium storing software of claim 37, further comprising instructions for causing a computer system to receive a designation, for each analyte that is above or below a specified normal limit, of an assigned relationship score that reflects the strength of the relationship between the analyte and the biomarker; generate a total assigned relationship score by summing each of the assigned relationship scores; and generate a functional need score using the total assigned relationship score.
